(19) United States
(12) Patent Application Publication
(10) Pub. No.: US 2025/0025444 A1
(43) Pub. Date: Jan. 23, 2025

(54) TREATMENT OF NEURODEGENERATIVE DISEASES VIA ADMINISTRATION OF BUNTANETAP AND A PHOSPHODIESTERASE INHIBITOR

(71) Applicants: ANNOVIS BIO, INC., Berwyn, PA (US); THE TRUSTEES OF COLUMBIA UNIVERSITY IN THE CITY OF NEW YORK, New York, NY (US)

(72) Inventors: Maria Maccecchini, West Chester, PA (US); Ottavio Arancio, New York, NY (US); Erica Acquarone, New York, NY (US)

(73) Assignees: ANNOVIS BIO, INC., Berwyn, PA (US); THE TRUSTEES OF COLUMBIA UNIVERSITY IN THE CITY OF NEW YORK, New York, NY (US)

(21) Appl. No.: 18/900,035

(22) Filed: Sep. 27, 2024

Related U.S. Application Data
(60) Provisional application No. 63/339,912, filed on May 9, 2022.

(51) Int. Cl. A61K 31/407; A61K 31/47; A61K 31/4985; A61K 31/506; A61K 31/519; A61K 31/53; A61P 25/16; A61P 25/28

(52) U.S. Cl. CPC A61K 31/407; A61K 31/47; A61K 31/4985; A61K 31/506; A61K 31/519; A61K 31/53; A61P 25/16; A61P 25/28

(57) ABSTRACT

The invention relates to methods and pharmaceutical compositions effective for treating, inhibiting, preventing, slowing, or delaying the onset of a neurodegenerative disease in mammals (e.g., humans) via the co-administration of an effective amount of a compound selected from the group consisting of Formula (I), Formula (II), Formula (III) or Formula (IV) or pharmaceutically acceptable salts thereof and a phosphodiesterase inhibitor (e.g., a PDE 5 inhibitor). In certain embodiments, the mammal is a healthy human, or a human experiencing cognitive dysfunction with or without hypertension.

This application claims the benefit of U.S. Provisional Application No. 63/633,912, filed on Apr. 15, 2024, hereby incorporated by reference.

### FIELD OF THE INVENTION

The present patent application concerns methods of inhibiting, preventing, or treating neurodegenerative diseases, pulmonary hypertension, erectile dysfunction, and benign prostatic hyperplasia (BPH) via administration of buntanetap, or related compounds, together with a phosphodiesterase inhibitor.

### BACKGROUND OF THE INVENTION

High iron levels increase the translation of neurotoxic aggregating proteins, leading, e.g., to impairment of axonal transport, inflammation, nerve cell death, and cognitive and motor function impairments.

For unknown reasons, in a “sick” brain, the level of iron is high, which induces iron regulatory protein 1 to release mRNAs coding for neurotoxic aggregating proteins causing upregulation of translation and synthesis, leading to overproduction of neurotoxic aggregating proteins in the sick brain.

Buntanetap is a translational inhibitor of multiple neurotoxic aggregating proteins.

Cyclic adenosine monophosphate (cAMP) and cyclic guanosine monophosphate (cGMP) are nucleotide biological second messengers that regulate various biological processes, including, e.g., blood flow, cardiac muscle contraction, cell differentiation, neural transmission, glandular secretion, and gene expression. Intracellular receptors for these molecules include cyclic nucleotide phosphodiesterases, cyclic nucleotide dependent protein kinases (PGK), and cyclic nucleotide-gated channels.

Phosphodiesterases (“PDEs”) are a large group of enzymes that are expressed in a variety of tissues, including, e.g., brain, smooth muscle, lung, platelets, and kidneys. PDEs catalyze the hydrolysis of 3′,5′-cyclic nucleotides to the corresponding 5′ monophosphates and degrade cAMP and cGMP. Some PDEs specifically bind to cAMP (PDE 4, PDE 7, and PDE 8). Others, (PDE 5, PDE 6, and PDE 9) are highly specific for cGMP. The remaining PDEs (PDE 1, PDE 2, PDE 3, PDE 10, and PDE 11) have mixed specificity.

Phosphodiesterase inhibitors are drugs that inhibit phosphodiesterases.

Phosphodiesterase 5 inhibitors (“PDE 5 inhibitors”) are cyclic guanosine 3′,5′-monophosphate type five cGMP PDE inhibitors. PDE 5 inhibitors increase cGMP levels by inhibiting the degradative action of PDE 5 on cGMP. The increase in cGMP level enhances phosphorylation of the transcription factor and memory-affecting molecule cAMP-responsive element binding (CREB) through activation of cGMP-dependent-protein kinases.

PDE inhibitors are not currently approved by US FDA for treatment of neurodegenerative diseases.

### SUMMARY OF THE INVENTION

It is an object of the invention to provide a treatment for neurodegenerative diseases, pulmonary hypertension, erectile dysfunction, and benign prostatic hyperplasia.

It is a further object of the invention to provide a treatment for neurodegenerative disorders which present as misfolding, aggregation and accumulation of neurotoxic proteins in the brain.

It is a further object of the invention to prevent, slow, or delay the development of neurodegenerative diseases that result in neuronal cell death.

It is an additional object of the invention to prevent, slow, or delay the development of cognitive impairment.

It is an additional object of the invention to prevent, slow, or delay the development of motor function impairment.

It is a further object of the invention to provide treatment for pulmonary hypertension.

It is an additional object of the invention to provide treatment for erectile dysfunction.

It is an additional object of the invention to provide treatment for benign prostatic hyperplasia.

In accordance with the above objects and others, the invention is directed in part to a method of treating a disease via administration of (i) buntanetap, or a compound that is similar to buntanetap as described herein, together with (ii) a phosphodiesterase inhibitor to a human in need thereof, wherein an administered amount of at least one of these agents is at least 2 times lower than the amount of that agent that would be required to provide any meaningful therapeutic effect if that agent were to be administered alone (i.e., as a monotherapy, without the other agent). Yet, the therapeutic effect provided by administration of (i) buntanetap, or the compound that is similar to buntanetap, together with (ii) the phosphodiesterase inhibitor is (a) greater than the therapeutic effect, if any, provided by administration of the same amount of buntanetap, or the compound that is similar to buntanetap, alone, and (b) is greater than the therapeutic effect, if any, provided by administration of the same amount of the phosphodiesterase inhibitor alone.

The invention is also directed in part to a method of treating a disease via administration of (i) buntanetap, or a compound that is similar to buntanetap, together with (ii) a phosphodiesterase inhibitor to a human in need thereof, wherein the buntanetap and the phosphodiesterase inhibitor are administered in amounts that are synergistic.

The invention is further directed to a method of treating a disease via the administration of an amount of buntanetap, or the compound that is similar to buntanetap, together with an amount of a phosphodiesterase inhibitor to a human patient in need thereof, wherein the amount of the phosphodiesterase inhibitor is subtherapeutic when administered without buntanetap, or the compound that is similar to buntanetap, but is therapeutic when administered with the amount of buntanetap, or the compound that is similar, to buntanetap.

The invention is also directed to a method of treating a disease via the administration of an amount of buntanetap, or the compound that is similar to buntanetap, together with an amount of a phosphodiesterase inhibitor to a human patient in need thereof, wherein the amount of buntanetap, or the compound that is similar to buntanetap, is subtherapeutic when administered without the phosphodiesterase inhibitor, but is therapeutic when administered with the amount of the phosphodiesterase inhibitor.

The administration of (i) buntanetap, or a compound that is similar to buntanetap, together with (ii) a phosphodiesterase inhibitor may provide a therapeutic effect that is at least 4 times greater than the therapeutic provided by administration of the phosphodiesterase inhibitor alone. In certain embodiments, improvement provided by administration of buntanetap, or the compound that is similar to buntanetap, together with the phosphodiesterase inhibitor is 4 to 10 times greater than the improvement provided by administration of the phosphodiesterase inhibitor alone.

The invention encompasses a method of treating a disease via administration of (i) buntanetap, or a compound that is similar to buntanetap as described herein, together with (ii) a phosphodiesterase inhibitor to a human in need thereof, wherein an administered amount of at least one of these agents is at least 5, 8 or 10 times lower than the amount of that agent that would be required to provide any meaningful therapeutic effect if that agent were to be administered alone.

The methods of the present invention may therefore allow for (i) administration of (a) a lower dose of buntanetap, or the compound that is similar to buntanetap, and/or (b) a lower dose of the phosphodiesterase inhibitor, (ii) a reduction in incidence and/or severity of adverse effect(s), and/or (iii) an increased efficacy of (a) the buntanetap, or the compound that is similar to buntanetap, and/or (b) the phoshpodiasterase inhibitor.

For example, in some of the embodiments, the invention is directed to a method of treating a disease via administration of (i) buntanetap, or a compound that is similar to buntanetap as described herein, together with (ii) a phosphodiesterase inhibitor to a human in need thereof, wherein buntanetap, or the compound that is similar to buntanetap, is administered in an amount that per day is less than 1 mg, less than 0.9 mg or less than 0.8 mg. In some of these embodiments, the administered amount of buntanetap, or the compound that is similar to buntanetap, may e.g., be from about 0.01 mg to about 0.7 mg, and could be administered orally, parenterally or transdermally.

The invention is also directed in part to a method of treating a disease via administration of (i) buntanetap, or a compound that is similar to buntanetap as described herein, together with (ii) a phosphodiesterase inhibitor to a human in need thereof, wherein the phosphodiesterase inhibitor is administered in an amount that per day is less than 5 mg, less than 4 mg or less than 3 mg. In some of these embodiments, the administered amount of the phosphodiesterase inhibitor may e.g., be from about 0.01 mg to about 2 mg, and could be administered orally, parenterally or transdermally.

The invention is also directed in part to a method of treating a disease via administration of (i) buntanetap, or a compound that is similar to buntanetap as described herein, together with (ii) a phosphodiesterase inhibitor to a human in need thereof, wherein the phosphodiesterase inhibitor is administered in an amount that is lower than an amount of buntanetap, or the compound that is similar to buntanetap.

Diseases that could be treated by the methods of the invention include, e.g., neurodegenerative disorders (e.g., Alzheimer's disease, dementia with Lewy bodies, frontotemporal dementia, other forms of dementia, chronic traumatic encephalopathy, tauopathies, alpha-synucleopathies, Prion's disease, Down Syndrome, Huntington's disease, multiple sclerosis, Amyloid Lateral Sclerosis), dementias, Parkinson's disease, pulmonary hypertension, erectile dysfunction, and benign prostatic hyperplasia.

Depending on the disease being treated, the therapeutic effect provided by the administration in accordance with the methods of the invention may include, e.g., a reduction of and/or severity of a symptom of a neurodegenerative disease, an improvement in cognitive function, an improvement in motor function, a reduction in blood pressure in arteries in the lungs and heart, a reduction of a symptom of an erectile dysfunction, and a reduction of a symptom of benign prostatic hyperplasia.

The invention is directed in part to a method of treating a neurodegenerative disease via the administration of (i) buntanetap, or the compound that is similar to buntanetap, together with (ii) a phosphodiesterase inhibitor to a human patient in need thereof, wherein the neurodegenerative disease is Alzheimer's disease, and the therapeutic effect is an improvement in mental status.

The invention is also directed in part to a method of treating a neurodegenerative disease via the administration of (i) buntanetap, or the compound that is similar to buntanetap, together with (ii) a phosphodiesterase inhibitor to a human patient in need, wherein the neurodegenerative disease is dementia (e.g., Parkinson's dementia, dementia with Lewy bodies, frontotemporal dementia, mild cognitive impairment, etc.), and the therapeutic effect is an improvement in cognition and mental status. These improvements in mental status may, e.g., be measured by a change in a score on ADAS-Cog11.

The invention is also directed in part to a method of treating a neurodegenerative disease via the administration of (i) buntanetap, or the compound that is similar to buntanetap, together with (ii) a phosphodiesterase inhibitor to a human patient in need thereof, wherein the neurodegenerative disease is Parkinson's disease, and the therapeutic effect is an improvement in motor function. The improvement in motor function may be measured, e.g., by a change in a score on MDS-UPDRS or a part thereof.

The invention is also directed in part to a method of treating Parkinson's disease via the administration of (i) buntanetap, or the compound that is similar to buntanetap, together with (ii) a phosphodiesterase inhibitor to a human patient in need, wherein the therapeutic effect are improvements e.g. in Postural Instability, Gait Difficulty, Tremors, or Cognition.

The invention is also directed in part to a method of treating an erectile dysfunction via the administration of (i) buntanetap, or the compound that is similar to buntanetap, together with (ii) a phosphodiesterase inhibitor to a human patient in need thereof. The improvement in erectile dysfunction may be measured, e.g., by a change in an International Index of Erectile Function score.

The invention is also directed in part to a method of treating benign prostatic hyperplasia via the administration of (i) buntanetap, or the compound that is similar to buntanetap, together with (ii) a phosphodiesterase inhibitor to a human patient in need thereof. The improvement in benign prostatic hyperplasia may be measured, e.g., by a change in an International Prostate Symptom Score.

The invention is also directed in part to a method for treating, inhibiting, preventing, slowing, or delaying the onset of a neurodegenerative disease comprising co-administering to the human:
(1) a therapeutically effective amount of a compound selected from the group consisting of Formula (I), Formula (II) and Formula (III):

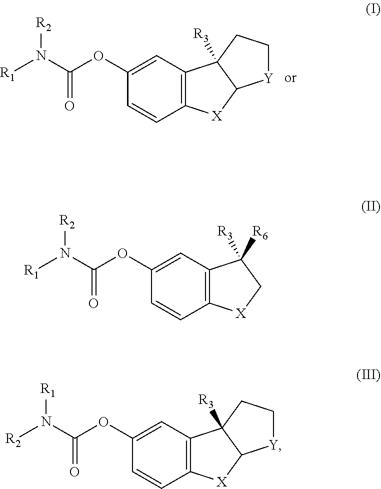

wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) a therapeutically effective amount of a phosphodiesterase inhibitor;
wherein the administration of the phosphodiesterase inhibitor with the compound selected from the group consisting of Formula (I), Formula (II) and Formula (III) provides a synergistic therapeutic effect.

The invention is also directed in part to a method for treating, inhibiting, preventing, slowing, or delaying the onset of a neurodegenerative disease comprising co-administering to the human:
(1) a therapeutically effective amount of a compound selected from the group consisting of Formula (I), Formula (II) and Formula (III):

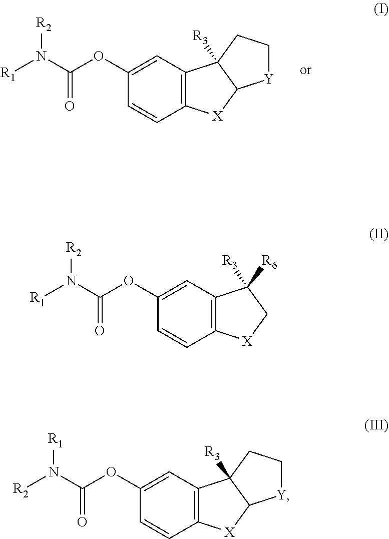

wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
   wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) a therapeutically effective amount of a phosphodiesterase inhibitor;
   wherein the phosphodiesterase inhibitor is a compound of Formula (V):

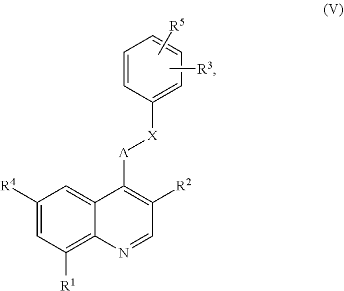

wherein A is 0 or N;
X is —(CH2)ₙ, C(O), S(O), or S(O)₂;
R₁ is hydrogen, C₁-C₆ alkyl, C₃-C₈ cycloalkyl, —NR⁷R⁸, —SR⁷, or heterocyclyl;
R₂ is —CH₂OR⁶ or —CO₂R⁸;
R³ is hydrogen or halogen;
R⁴ is —CN or halogen;
R⁵ is hydrogen or —OR⁶;
R⁶ is hydrogen, —C₁-C₆ alkyl, —C₃-C₈ cycloalkyl, or —C(O)R⁹;
R⁷ and R⁸ are each independently hydrogen, —C₁-C₆ alkyl, —C₃-C₈ cycloalkyl, or —C(O)R⁹, wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl; or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C₁-C₆ alkyl; and
R⁹ and R¹⁰ are each independently hydrogen, C₁-C₆ alkyl, or C₃-C₈ cycloalkyl; and n is 1, 2, or 3, or a pharmaceutically acceptable salt or tautomer thereof;
and the administration provides a synergistic therapeutic effect.

The invention is also directed in part to a method for treating, inhibiting, preventing, slowing, or delaying the onset of a neurodegenerative disease comprising co-administering to the human:
(1) a therapeutically effective amount of buntanetap or a pharmaceutically acceptable salt thereof; and
(2) a therapeutically effective amount of 4-[(3-Chloro-4-methoxybenzyl)amino]-8-cyclopropyl-3-(hydroxymethyl) quinoline-6-carbonitrile or a pharmaceutically acceptable salt thereof; wherein and the administration provides a therapeutic effect that is greater than a therapeutic effect provided by administration of buntanetap without 4-[(3-Chloro-4-methoxybenzyl)amino]-8-cyclopropyl-3-(hydroxymethyl) quinoline-6-carbonitrile and/or a therapeutic effect that is greater than a therapeutic effect provided by administration of 4-[(3-Chloro-4-methoxybenzyl)amino]-8-cyclopropyl-3-(hydroxymethyl) quinoline-6-carbonitrile without buntanetap. In certain embodiments, the administration of 4-[(3-Chloro-4-methoxybenzyl)amino]-8-cyclopropyl-3-(hydroxymethyl) quinoline-6-carbonitrile buntanetap provides a synergistic therapeutic effect.

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 0.9 mg together with a therapeutically effective amount of a phosphodiesterase inhibitor, wherein the phosphodiesterase inhibitor is a phosphodiesterase 5 inhibitor, and, after administration for at least three months, provides an increase in an ADAS-Cog score.

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 0.9 mg together with a therapeutically effective amount of a phosphodiesterase inhibitor, wherein the phosphodiesterase inhibitor is a compound of Formula (V):

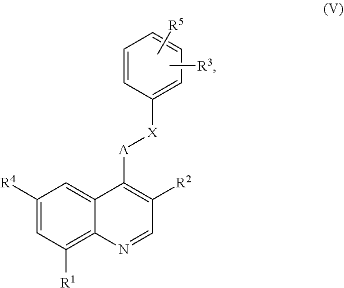

wherein A is 0 or N;
X is —(CH2)ₙ, C(O), S(O), or S(O)₂;
R₁ is hydrogen, C₁-C₆ alkyl, C₃-C₈ cycloalkyl, —NR⁷R⁸, —SR⁷, or heterocyclyl;
R₂ is —CH₂OR⁶ or —CO₂R⁸;
R³ is hydrogen or halogen;
R⁴ is —CN or halogen;
R⁵ is hydrogen or —OR⁶;
R⁶ is hydrogen, —C₁-C₆ alkyl, —C₃-C₅ cycloalkyl, or —C(O)R⁹;
R⁷ and R⁸ are each independently hydrogen, —C₁-C₆ alkyl, —C₃-C₈ cycloalkyl, or —C(O)R⁹, wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl; or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C₁-C₆ alkyl; and
R⁹ and R¹⁰ are each independently hydrogen, C₁-C₆ alkyl, or C₃-C₈ cycloalkyl; and n is 1, 2, or 3, or a pharmaceutically acceptable salt or tautomer thereof; and wherein the administration provides an increase in an ADAS-Cog11 score, as compared to an ADAS-Cog11 score at the start of the administration.

In certain embodiments, the compound selected from the group consisting of Formula (I), Formula (II) and Formula (III) is buntanetap or a pharmaceutically acceptable salt thereof, is administered orally once-a-day for at least three months at a dose of from about 0.01 mg to about 0.9 mg together with a therapeutically effective amount of a phosphodiesterase inhibitor, wherein the phosphodiesterase inhibitor is 4-[(3-Chloro-4-methoxybenzyl)amino]-8-cyclopropyl-3-(hydroxymethyl) quinoline-6-carbonitrile or a pharmaceutically acceptable salt thereof.

In certain embodiments, the compound selected from the group consisting of Formula (I), Formula (II) and Formula (III) is buntanetap, or a pharmaceutically acceptable salt thereof, is administered orally once-a-day for at least three months at a dose from about 0.01 mg to about 0.9 mg, the phosphodiesterase inhibitor is a phosphodiesterase 5 inhibitor, and, the administration for at least three months provides an increase in an ADAS-Cog11, as compared to the ADAS-Cog11 score at the start of the administration.

The invention is also directed in part to a method to inhibit, prevent or treat a neurodegenerative disease such as Alzheimer's disease via the administration of buntanetap, a compound that is similar to buntanetap as described herein, pharmaceutically acceptable salts and complexes thereof, together with a phosphodiesterase inhibitor (e.g., a PDE 5 inhibitor) to a human patient in need thereof, wherein the buntanetap, or the compound that is similar to buntanetap, potentiates a therapeutic effect of the phosphodiesterase inhibitor, and/or the a phosphodiesterase inhibitor potentiates a therapeutic effect of buntanetap, or the compound that is similar to buntanetap by at least 50% (e.g., by 100 to 900%).

The invention is also directed in part to a method of treating pulmonary hypertension via the administration of buntanetap, a compound that is similar to buntanetap as described herein, pharmaceutically acceptable salts and complexes thereof, together with a phosphodiesterase inhibitor (e.g., a PDE 5 inhibitor) to a human patient in need thereof, wherein the buntanetap, or the compound that is similar to buntanetap, potentiates a therapeutic effect of the phosphodiesterase inhibitor, and/or the a phosphodiesterase inhibitor potentiates a therapeutic effect of buntanetap, or the compound that is similar to buntanetap by at least 50% (e.g., by 100 to 900%).

The invention is also directed in part to a method of treating benign prostatic hyperplasia via the administration of buntanetap, a compound that is similar to buntanetap as described herein, pharmaceutically acceptable salts and complexes thereof, together with a phosphodiesterase inhibitor (e.g., a PDE 5 inhibitor) to a human patient in need thereof, wherein the buntanetap, or the compound that is similar to buntanetap, potentiates a therapeutic effect of the phosphodiesterase inhibitor, and/or the a phosphodiesterase inhibitor potentiates a therapeutic effect of buntanetap, or the compound that is similar to buntanetap by at least 50% (e.g., by 100 to 900%).

The invention is further directed in part to a method of treating a disease via the administration of buntanetap, or the compound that is similar to buntanetap, together a phosphodiesterase inhibitor (e.g., phosphodiesterase 5 inhibitor) to a human patient in need thereof in a weight ratio that provides a synergistic therapeutic effect, as compared to the administration of buntanetap without the phosphodiesterase inhibitor and/or administration of the phosphodiesterase inhibitor without buntanetap. In some of the embodiments, the synergistic ratio weight ratio is from 10 to 10000, from 10 to 5000, from 10 to 1000, from 10 to 500, or from 10 to 250.

The invention is also directed in part to a method of treating a neurodegenerative disease via the administration of buntanetap, or the compound that are similar to buntanetap, together with a phosphodiesterase inhibitor to a human patient in need thereof in amounts that provide a synergistic therapeutic effect, as compared to the administration of buntanetap without the phosphodiesterase inhibitor and/or administration of the phosphodiesterase inhibitor without buntanetap.

The invention is also directed in part to a method of treating pulmonary hypertension via the administration of buntanetap, or the compound that is similar to buntanetap, together with a phosphodiesterase inhibitor to a human patient in need thereof in amounts that provide a synergistic therapeutic effect, as compared to the administration of buntanetap without the phosphodiesterase inhibitor and/or administration of the phosphodiesterase inhibitor without buntanetap.

The invention is also directed in part to a method of treating erectile dysfunction via the administration of buntanetap, or the compound that is similar to buntanetap, together with phosphodiesterase inhibitor to a human patient in need thereof in amounts that provide a synergistic therapeutic effect, as compared to the administration of buntanetap without the phosphodiesterase inhibitor and/or administration of the phosphodiesterase inhibitor without buntanetap.

The invention is also directed in part to a method of treating benign prostatic hyperplasia via the administration of buntanetap, or the compound that is similar to buntanetap, together with phosphodiesterase inhibitor to a human patient in need thereof in amounts that provide a synergistic therapeutic effect, as compared to the administration of buntanetap without the phosphodiesterase inhibitor and/or administration of the phosphodiesterase inhibitor without buntanetap.

The invention is further directed in part to a method of treating a dementia via the administration of buntanetap, or the compound that is similar to buntanetap, together with a phosphodiesterase inhibitor to a human patient in need thereof in amounts that provide a synergistic therapeutic effect, as compared to the administration of buntanetap without the phosphodiesterase inhibitor and/or administration of the phosphodiesterase inhibitor without buntanetap.

The invention is also directed in part to a method to inhibit, prevent or treat a neurodegenerative disease such as Alzheimer's disease via the administration of buntanetap, a compound that is similar to buntanetap as described herein, pharmaceutically acceptable salts and complexes thereof, together with a phosphodiesterase inhibitor (e.g., a PDE 5 inhibitor), and one or more pharmaceutically acceptable excipients in a patient who does not have pulmonary hypertension, does not have erectile dysfunction and does not have benign prostatic hyperplasia.

The invention is also directed in part to a method to inhibit, prevent or treat a neurodegenerative disease such as Alzheimer's disease in a patient who has pulmonary hypertension via the administration of buntanetap, compounds that are similar to buntanetap as described herein, pharmaceutically acceptable salts and complexes thereof, together with a phosphodiesterase inhibitor.

It is a further object of the invention to inhibit, prevent, slow or reduce the overexpression of neurotoxic aggregating proteins in a patient who is experiencing pulmonary hypertension or general hypertension, who is not demonstrating symptoms of a neurological disorder or a neurodegenerative disease, or who is demonstrating symptoms of a neurological disorder or a neurodegenerative disease, comprising or consisting of administering to the human a therapeutically effective amount of buntanetap, active metabolites of buntanetap, therapeutically effective analogues of buntanetap, compounds that are similar to buntanetap as described herein, pharmaceutically acceptable salts and complexes thereof, together with a phosphodiesterase inhibitor in a therapeutic dose used to treat hypertension or in a subtherapeutic dose as compared to a dose of the a phosphodiesterase inhibitor used to treat hypertension. Examples of neurotoxic aggregating proteins are APP (amyloid precursor protein), Aβ (amyloid-β peptide, a fragment of APP), SOD (super oxide dismutase) proteins, Tau, alpha-synuclein (SNCA), transmissible spongiform encephalopathy (TSE) prions, TDP43, and huntingtin (HTT).

It is a further object of the invention to treat, inhibit, or slow the onset of neurological disorders or diseases such as Alzheimer's disease, other dementias (e.g., dementia with Lewy bodies, frontotemporal dementia, etc.), tauopathies, chronic traumatic encephalopathy, Parkinson's disease, alpha-synucleopathies, Prion's disease, Down Syndrome, Huntington's disease, Amyloid Lateral Sclerosis, multiple sclerosis, and neurodegenerative disorders in mammals (e.g., humans) comprising or consisting of administering to the human a therapeutically effective amount of buntanetap, active metabolites of buntanetap, therapeutically effective analogues of buntanetap, compounds that are similar to buntanetap as described herein, pharmaceutically acceptable salts and complexes thereof, together with a phosphodiesterase inhibitor, and one or more pharmaceutically acceptable excipients. In certain embodiments, buntanetap is administered orally in an amount from about 0.01 mg to about 50 mg, preferably on a once-a-day basis, and the phosphodiesterase inhibitor is administered in an amount from about 0.01 mg to about 25 mg, from about 0.5 mg to about 15 mg, or from about 1 mg to about 4 mg, preferably on a once-a-day basis.

In the methods of the invention, buntanetap, or the compound that is similar to buntanetap, and the phosphodiesterase inhibitor may be administered as free bases, pharmaceutically acceptable salts, metabolites, prodrugs, complexes, and mixtures thereof by the same or different route of administration and at the same or different times.

In certain embodiments, buntanetap is administered in an amount from about 0.01 mg to about 120 mg, preferably on a once-a-day basis. Thus, in certain embodiments, a dose of buntanetap may, e.g., be 0.01 mg, 0.02 mg, 0.05 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg, and numbers in between these numbers. In certain preferred embodiments, buntanetap is administered orally in a dose from about 0.1 mg to about 5 mg, from about 0.1 mg to about 2 mg or from about 0.1 mg to about 0.9 mg. In other preferred embodiments, the buntanetap dose is administered intravenously in an amount from about 0.01 to about 0.25 mg/day. In other preferred embodiments, the buntanetap dose is administered intraperitoneally/intramuscularly (IP/IM) in a dose from about 00.3 mg/day to about 0.7 mg/day. In certain preferred embodiments, the buntanetap is administered together with any phosphodiesterase inhibitor as described further herein in following paragraphs.

In certain embodiments, a phosphodiesterase inhibitor is administered in an amount from about 0.02 mg to about 100 mg, preferably on a once-a-day basis. A dose of the phosphodiesterase inhibitor may, e.g., be 0.02 mg, 0.05 mg, 0.1 mg, 0.2 mg, 0.5 mg, 0.75 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, and numbers in between these numbers, and all further integers to about 2000 mg. The phosphodiesterase inhibitor may, e.g., be administered orally, parenterally, sublingually, via suppository, nasally, topically, transdermally, or via implant under the skin.

In certain embodiments of each of the methods described above, the oral pharmaceutical composition includes from about 0.1 mg to about 5 mg buntanetap or a pharmaceutically acceptable salt thereof, the IP/IM pharmaceutical composition includes from about 0.03 to about 0.7 mg buntanetap or a pharmaceutically acceptable salt thereof, and the intravenous (IV) pharmaceutical formulation includes from about 0.01 to about 0.25 mg buntanetap or a pharmaceutically acceptable salt thereof.

In certain embodiments of each of the methods described above, to minimize incidence of side effects, buntanetap or a pharmaceutically acceptable salt thereof is administered multiple times during the day or in a controlled or sustained release formulation that releases buntanetap or a pharmaceutically acceptable salt thereof over 4 to 24 hours, preferably over 8 to 24 hours or 12 to 24 hours.

In certain preferred embodiments of the methods described herein, peak plasma circulating levels of buntanetap in humans range, e.g., from about 0.01 ng/mL to about 200 ng/mL, in certain embodiments from about 0.2 ng/mL to about 20 ng/mL, and more preferably from about 0.37 ng/mL to about 12 ng/mL, and any numbers or integers in between. In certain preferred embodiments, the peak plasma circulating level is reached within about 1 to 3 hours after administration of buntanetap to humans. In certain embodiments, the steady-state plasma concentration of buntanetap in brain is about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 ng/mL. In certain embodiments, the half-life of buntanetap in cerebrospinal fluid after administering is about 12 hours, and the half-life of buntanetap in plasma after administering is about 5 hours. In certain embodiments, the administration of buntanetap to humans results in a brain level of buntanetap that range from about 4 to about 10 times the plasma level of buntanetap in those patients. In certain embodiments, the concentration of buntanetap in the brain of humans is from about 0.8 ng/g to about 304 ng/g, in certain embodiments from about 0.3 ng/g to about 100 ng/g.

With respect to each of the methods described above, buntanetap, a pharmaceutically acceptable salt thereof, analogues or similar compounds as described herein may be administered, e.g., orally, parenterally, sublingually, via suppository, nasally, topically, transdermally, or via implant under the skin.

The invention is further directed in part to a method for preventing, treating, inhibiting, or slowing a neurodegenerative disease such as Alzheimer's disease in a healthy human who is at risk of developing such a neurodegenerative disease comprising administering to the human a PDE 5 inhibitor together with a compound selected from the group consisting of Formula (I), Formula (II), Formula (III) and Formula (IV):

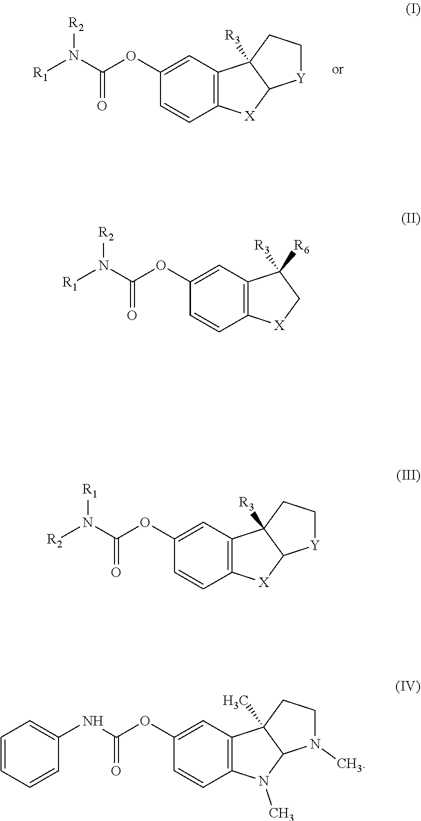

In Formula (I) and Formula (II), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen, C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4. In certain embodiments, the method includes the use of a second therapeutic active agent to treat, prevent, delay, or slow the onset of neurodegenerative disease such as Alzheimer's disease.

In certain embodiments, the compound selected from the group consisting of Formula (I), Formula (II), Formula (III) and Formula (IV) is devoid of any significant acetylcholinesterase (ACHE) inhibitory activity and devoid of any butyrylcholinesterase (BCHE) activity.

In certain embodiments, the compound of Formula (I) and Formula (II) is the a (+)-enantiomer. In other embodiments, the compound of Formula (I) and Formula (II) is a (−)-enantiomer.

In certain embodiments, the compound of Formula (I) is buntanetap or its active metabolites.

In preferred embodiments, the compound is buntanetap of Formula IV as follows:

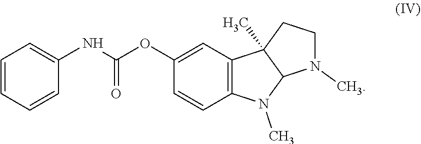

In Formula (III), R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋₈ alkenyl, C₂₋₈ alkynyl, or aralkyl, and Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl.

The invention is further directed in part to a method for treating, preventing, delaying, or slowing a neurodegenerative disease such as Alzheimer's disease comprising administering to the human a compound selected from the group consisting of Formula (I), Formula (II), Formula (III) or Formula (IV) together with a phosphodiesterase 5 inhibitor.

The phosphodiesterase inhibitors are considered for purposes of this invention to encompass Phosphodiesterase 5 (“PDE 5”) 5 inhibitors, and include, e.g., sildenafil, vardenafil, tadalafil, and avanafil, 4-[(3-Chloro-4-methoxybenzyl)amino]-8-cyclopropyl-3-(hydroxymethyl) quinoline-6-carbonitrile, and other PDE 5 inhibitors.

PDE 5 inhibitors are a type of targeted therapy used, e.g., to treat people with pulmonary hypertension (PH). Targeted therapies slow the progression of PH and may even reverse some of the damage to the heart and lungs.

PDE 5 inhibitors are also used to treat erectile dysfunction (“ED”). This is because the body has the same type of cells in the blood vessels of the lungs as the blood vessels of the penis.

Sildenafil, vardenafil, tadalafil, and avanafil are indicated for the treatment of men with ED. Sildenafil, the first PDE 5 inhibitor, was introduced in 1998. More than 20 million men were treated with sildenafil in its first 6 years on the market. In 2003, vardenafil was approved, offering patients an alternative option. Tadalafil followed several months later and was also approved in 2003. Nicknamed the “weekend pill,” tadalafil's 36-hour effectiveness offered patients more spontaneity. Several years after the introduction of tadalafil on the market, researchers toyed with the idea of a chronic, low-dose formulation to further enhance spontaneity. In 2008, Eli Lilly obtained FDA approval for the once-daily administration of tadalafil. In 2010, a 10-mg oral disintegrating tablet (ODT) formulation of vardenafil (Staxyn®) was introduced; this ODT discreet formulation is considered more convenient to administer.

In October 2011, tadalafil (Cialis®) was also approved to treat benign prostatic hyperplasia (BPH) with or without ED.

Avanafil (Stendra®) was approved in April 2012, offering an onset of action as early as 15 minutes after administration and further expanding treatment options for men with ED.

PDE 5 inhibitors may also be used in females, e.g., to treat female sexual arousal disorder (FSAD), which is a type of sexual dysfunction characterized by low sex drive (libido).

PDE 5 inhibitors of the invention include compounds of Formula (V):

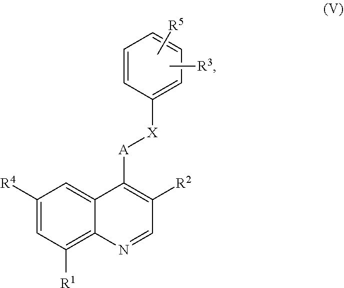

wherein A is O or N;
X is —(CH2)ₙ, C(O), S(O), or S(O)₂;
R₁ is hydrogen, C₁-C₆ alkyl, C₃-C₈ cycloalkyl, —NR⁷R⁸, —SR⁷, or heterocyclyl;
R₂ is —CH₂OR⁶ or —CO₂R⁸;
R³ is hydrogen or halogen;
R⁴ is —CN or halogen;
R⁵ is hydrogen or —OR⁶;
R⁶ is hydrogen, —C₁-C₆ alkyl, —C₃-C₈ cycloalkyl, or —C(O)R⁹;
R⁷ and R⁸ are each independently hydrogen, —C₁-C₆ alkyl, —C₃-C₈ cycloalkyl, or —C(O)R⁹, wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl; or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C1-C6 alkyl; and
R⁹ and R¹⁰ are each independently hydrogen, C₁-C₆ alkyl, or C₃-C₈ cycloalkyl; and n is 1, 2, or 3,
or a pharmaceutically acceptable salt or tautomer thereof.

Compounds of Formula V include compounds of Formula (V-1):

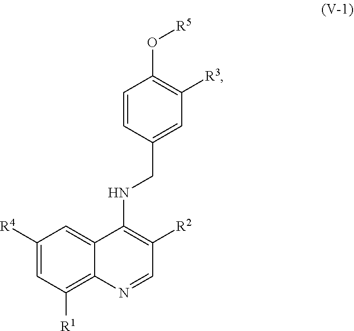

wherein
R¹ is hydrogen, C₁-C₆ alkyl, C₃-C₈ cycloalkyl, —NR⁷R⁸, —SR⁷, or heterocyclyl;
R² is —CH₂OR⁶ or —CO₂R⁸;
R³ is hydrogen or halogen;
R⁴ is —CN or halogen;
R⁶ is hydrogen, —C₁-C₆ alkyl, —C₃-C₈ cycloalkyl, or —C(O)R⁹;
R⁷ and R⁸ are each independently hydrogen, —C₁-C₆ alkyl, —C₃-C₈ cycloalkyl, or —C(O)R⁹,
wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl; or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C₁-C₆ alkyl; and
R⁹ and R¹⁰ are each independently hydrogen, C₁-C₆ alkyl, or C₃-C₈ cycloalkyl, or a pharmaceutically acceptable salt or tautomer thereof.

Compounds of Formula V include compounds of Formula (V-1a):

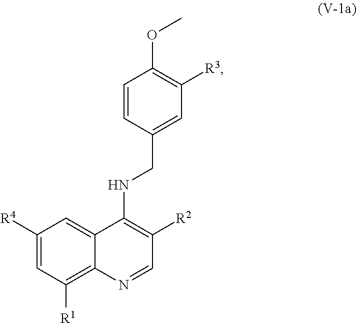

wherein
R¹ is hydrogen, C₁-C₆ alkyl, C₃-C₈ cycloalkyl, —NR⁷R⁸, —SR⁷, or heterocyclyl;
R² is —CH₂OR⁶ or —CO₂R⁸;
R³ is hydrogen or halogen;
R⁴ is —CN or halogen;
R⁷ and R⁸ are each independently hydrogen, —C₁-C₆ alkyl, —C₃-C₈ cycloalkyl, or —C(O)R⁹, wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C₁-C₆ alkyl; and
R⁹ and R¹⁰ are each independently hydrogen, C₁-C₆ alkyl, or C₃-C₈ cycloalkyl,
or a pharmaceutically acceptable salt or tautomer thereof.

Compounds of Formula V also include compounds of Formula (V-1a1):

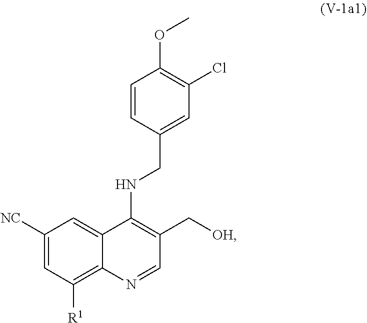

wherein
R¹ is hydrogen, C₁-C₆ alkyl, C₃-C₈ cycloalkyl, —NR⁷R⁸, —SR⁷, or heterocyclyl;
R⁷ and R⁸ are each independently hydrogen, —C₁-C₆ alkyl, —C₃-C₈ cycloalkyl, or —C(O)R⁹, wherein the C₁-C₆ alkyl or C₃-C₈ cycloalkyl are optionally substituted with —C₁-C₆ alkyl, —C₃-C₈ cycloalkyl, —NR⁹R¹⁰, —SR⁹, or heterocyclyl or, R⁷ and R⁸ together with the nitrogen atom to which they are attached form a 3 to 8-membered heterocycle, wherein any one of the ring carbon atoms is optionally replaced with a heteroatom, and wherein the heterocycle is optionally substituted with C₁-C₆ alkyl; and
R⁹ and R¹⁰ are each independently hydrogen, C₁-C₆ alkyl, or C₃-C₈ cycloalkyl,
or a pharmaceutically acceptable salt or tautomer thereof.

In certain embodiments, a compound of Formula (V-1a1) is 4-[(3-Chloro-4-methoxybenzyl)amino]-8-cyclopropyl-3-(hydroxymethyl) quinoline-6-carbonitrile:

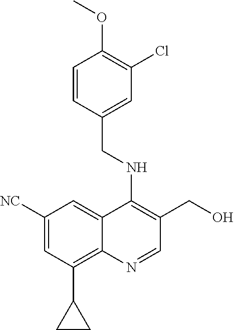 [SMILES: COc1ccc(CNc2c(CO)cnc3c(C4CC4)cc(C#N)cc23)cc1Cl]

or a pharmaceutically acceptable salt or tautomer thereof.

In certain embodiments, the methods of the present invention are practiced using phosphodiesterase inhibitors, known to those skilled in the art, such as those described in U.S. Pat. Nos. 9,974,782; 10,626,113; 10,899,756; 11,851,427; and WO Publication No. WO 2009/124,119, all of which are incorporated by reference in their entireties.

In certain embodiments, the methods of the present invention are practiced using phosphodiesterase inhibitors described in Fiorito et al., Synthesis of quinoline derivatives: Discovery of a potent and selective phosphodiesterase 5 inhibitor for the treatment ofAlzheimer's disease, European Journal of Medicinal Chemistry 60 (2013) 285-294, and Acquarone et al., Synaptic and memory dysfunction induced by tau oligomers is rescued by upregulation of the nitric oxide cascade, Molecular Neurodegeneration, 2019, both herein incorporated by reference in their entireties.

In certain embodiments, the compounds are administered in accordance with the formulations and methods of the invention to a patient who is already presenting with symptoms of a neurological disease. In other embodiments, the compounds are administered in accordance with the formulations and methods of the invention to a patient who is already presenting with pulmonary hypertension. In certain embodiments, the compounds are administered in accordance with the formulations and methods of the invention to a patient who is already presenting with symptoms of a neurological disease and pulmonary hypertension. In yet other embodiments, the compounds are administered in accordance with the formulations and methods of the invention to a patient (human subject) who is not presenting with any neurological or hypertensive problems, but, e.g., may be at risk for future neurological and/or hypertensive problems.

In the methods of the invention, the compounds are administered either separately or together via a route selected from the group consisting of orally, parenterally, sublingually, via suppository, nasally, topically, transdermally, intravenously, subcutaneously, intraperitoneally and via implant under the skin. In certain embodiments, the compound is administered chronically to treat, prevent, delay, or slow a neurodegenerative disease such as Alzheimer's disease in a mammal, e.g., a human. In certain embodiments, the neurological disorders or diseases encompass but are not limited to Alzheimer's disease, tauopathies, chronic traumatic encephalopathy, dementia (e.g., frontotemporal dementia, dementia with Lewy bodies, etc.), Parkinson's disease, an alpha-synucleopathy, Prion's disease, Down Syndrome, Huntington's disease, Amyloid Lateral Sclerosis, multiple sclerosis or another neurodegenerative disorder.

In certain preferred embodiments, the co-administered compounds are buntanetap and a PDE 5 inhibitor, and the buntanetap is administered in an amount as set forth in this disclosure, along with the PDE 5 inhibitor.

In certain embodiments, the compounds (e.g., buntanetap and a PDE 5 inhibitor) are co-administered to a mammal (e.g., human) who is symptomatic for pulmonary hypertension, e.g., experiencing decreased blood flow to the pulmonary arteries or hypertensive in general. In certain embodiments, the mammal (e.g., human subject or human patient) is demonstrating symptoms of a neurological disorder or a neurodegenerative disease. In certain embodiments, the mammal (e.g., human subject or human patient) is not demonstrating symptoms of a neurological disorder or a neurodegenerative disease.

In certain embodiments, the buntanetap or a pharmaceutically acceptable salt thereof is administered (i) orally in an amount from about 0.1 mg to about 12 mg on a once-a-day basis; (ii) intravenously in an amount from about 0.01 mg to about 2.5 mg/day; or (ii) intraperitoneally/intramuscularly (IP/IM) in a dose from about 0.03 to about 7 mg/day. The buntanetap or a pharmaceutically acceptable salt thereof is administered, e.g., orally in an amount from about 0.1 mg to about 8 mg on a once-a-day basis. The peak plasma circulating levels of buntanetap in humans range, e.g., from about 0.1 ng/mL to about 40 ng/mL.

In certain embodiments, the compound of Formulas (I), (II), and (III), and the phosphodiesterase inhibitor (e.g., PDE (5) inhibitor(s)) are administered separately but such that they provide overlapping therapeutic effects.

In certain embodiments, the invention is further directed to a pharmaceutical composition, comprising
(1) a therapeutically effective amount of a compound selected from the group consisting of Formula (I), Formula (II) and Formula (III):

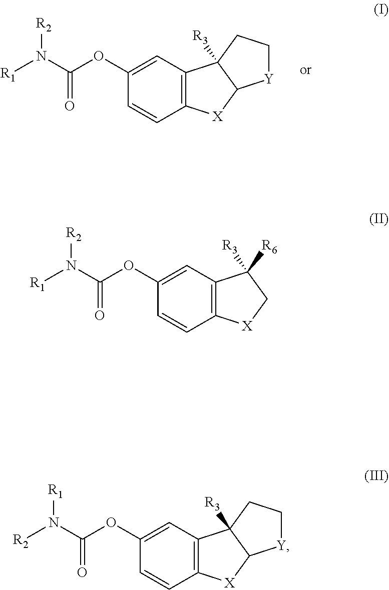

wherein,
in Formula (I) and Formula (II),
   R₁ and R₂ are, independently, hydrogen, branched or straight chain C₁-C₈ alkyl, substituted or unsubstituted aryl, heteroaryl, or aralkyl;
   R₃ is branched or straight chain C₁-C₄ alkyl or heteroalkyl or C₄-C₈ alkyl or heteroalkyl, or substituted or unsubstituted aryl;
   X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
   R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
   R₁ and R₂ are, independently, hydrogen, branched or straight chain C₁-C₈ alkyl, substituted or unsubstituted aryl, heteroaryl, or aralkyl;
   R₃ is branched or straight chain C₁-C₄ alkyl or heteroalkyl or C₄-C₈ alkyl or heteroalkyl, or substituted or unsubstituted aryl;
   X is NR₅, wherein R₅ is C₂₋₈ alkenyl, C₂₋₈ alkynyl, or aralkyl;
   Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) a therapeutically effective amount of a compound which is a PDE 5 inhibitor; and
(3) at least one pharmaceutically acceptable excipient. In certain preferred embodiments, the compound of Formula (III) is the substantially pure (−)-enantiomer. In certain embodiments, the compound is buntanetap of Formula (IV),

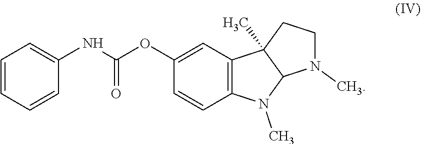 [SMILES: CN1CC[C@]2(C)c3cc(OC(=O)Nc4ccccc4)ccc3N(C)C12]

wherein the compound of Formula (IV) is the substantially pure (+)-enantiomer, or a pharmaceutically acceptable salt thereof. In certain embodiments, the buntanetap or a pharmaceutically acceptable salt thereof is in an amount from about 0.1 mg to about 20 mg, and more preferably from about 0.1 mg to about 5 mg, and the PDE 5 inhibitor is 4-[(3-Chloro-4-methoxybenzyl)amino]-8-cyclopropyl-3-(hydroxymethyl) quinoline-6-carbonitrile.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e. to at least one) of the grammatical objects of the article. By way of example, “an element” means one element or more than one element.

The term “about” will be understood by persons of ordinary skill in the art and will vary to some extent on the context in which it is used. In the context of the present application, the term “about” means a value within 20% (20%) of the value recited immediately after the term “about,” including the value equal to the upper limit (i.e., +20%) and the value equal to the lower limit (i.e., −20%) of this range. For example, the phrase “about 100” encompasses any numeric value that is between 80 and 120, including 80 and 120.

The term “a phosphodiesterase inhibitor” means a drug that inhibits a phosphodiesterase enzyme in mammals.

As used herein, the terms “buntanetap” and “Posiphen” are used interchangeably to refer to (3aR)-1,3a,8-trimethyl-1,2,3,3a,8,8a-hexahydropyrrolo[2,3-b]indol-5-yl phenylcarbamate or a salt thereof.

As used herein, the term “APP” refers to amyloid precursor protein.

As used herein, the term “Aβ” refers to Abeta or amyloid beta or amyloid-β peptide. For purposes of the present invention, these terms are considered to be synonymous.

For purposes of the present invention, a “buntanetap-type” drug or “the compound that is similar to buntanetap” encompasses Formula (I), Formula (II), Formula (III) or Formula (IV).

As used herein, the term “HTT” refers to huntingtin or the Huntington protein.

As used herein, “TDP43” refers to the TAR-DNA binding protein TDP43.

As used herein, “C₉orf72” refers to the C₉orf72 protein found in many regions of the brain.

As used herein, “efficacy” is the capacity of a drug (i.e., buntanetap and a phosphodiesterase activity) to produce a therapeutic effect.

As used herein, the term “neurotoxic aggregating protein” refers to a protein or family of proteins that has neurotoxic effect upon accumulating in a tissue of the brain, such as the brain tissue. Non-limiting examples of neurotoxic aggregating proteins are APP, Aβ, SOD1, SNCA, NAC, TSE amyloid plaque, HTT, Tau, TDP43 and C9orf72.

As used herein, the terms “protein”, “peptide” and “polypeptide” are used interchangeably and refer to a compound comprised of amino acid residues covalently linked by peptide bonds.

By the term “specifically binds,” as used herein, means a molecule, such as an antibody or a small molecule, which recognizes and binds to another molecule or feature, but does not substantially recognize or bind other molecules or features in a sample.

The term “inhibit” as used herein means to reduce a molecule, a reaction, an interaction, a gene, an mRNA, and/or a protein's expression, stability, function, or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function, and activity, e.g., antagonists.

Depending on the disease that is treated, the “therapeutic effect” provided by the methods of the present invention includes, e.g., an improvement in mental function, as determined by a change in a score on a scale for mental function assessment; an inhibition of a phosphodiesterase activity, a reduction in pulmonary hypertension, a reduction in nocturnal urination, an improvement in sexual function, a reduction in plasma Total Tau level; a decrease in the production of amyloid beta (Aβ); a decrease in the production of tau; a decrease in the production of alpha-Synuclein (αSYN); a decrease in the production of TDP43; and a combination of two or more of any of the foregoing.

In the context of the invention, the “improvement in mental function” may be ascertained by a change in a score on a test known to one of ordinary skill in the art, including e.g., Alzheimer's Disease Assessment Scale-Cognitive Subscale (e.g., “ADAS-Cog 11”, “ADAS-Cog 12”, “ADAS-Cog 13”, “ASAS-Cog 14”), a Mini-Mental State Examination (MMSE), a Montreal Cognitive Assessment (MoCA), Wechsler Intelligence Test, after 3 months of administration, as compared to a score at the start of administration, which is used as a baseline.

MMSE is a test of mental function that contains 30 questions and assesses six areas of metal abilities: orientation of time and place, attention and concentration, short-term memory recall, language skills, visuospatial abilities, and ability to understand and follow instructions. Scores on the MMSE range from 0 to 30, with scores of 25 or higher being traditionally considered normal; scores less than 10 generally indicating severe impairment; and scores between 10 and 20 indicating moderate dementia. People with early-stage Alzheimer's disease tend to score in the 20 to 25 range.

MoCA is a test used to detect mild cognitive decline and early signs of dementia. The MoCA test examines seven domains (aspects) of cognitive function with a total of eleven different exercises and tasks: executive and visuospatial function, naming, attention, language, abstraction, delayed recall, and orientation. The maximum score on MoCA is 30 points. A score between 18 and 25 points indicates mild cognitive impairment. A score between 10 and 17 points indicates moderate cognitive impairment. A score under 10 points indicates severe cognitive impairment.

ADAS-Cog is a cognitive rating scale, which was designed to target the cognitive and behavioral domains known to be affected in Alzheimer disease, including memory, language, orientation, construction, and planning of simple designs, and completed simple goal-oriented behaviors. ADAS-Cog 11 includes 11 items assessing cognitive function. The domains include memory, language, praxis, and orientation. There are 70 possible points, 48 for the first 9 items, and 22 for the last two items, word recall and recognition.

The ADAS-Cog-12 includes all ADAS-Cog-11 items as well as a delayed word recall task. During this task, after a period of time delay, a subject is given three trials to recall as many of the ten words from the Word Recall task as possible. The number of words not recalled (errors) is added to the score from the other 11 tasks, giving a final ADAS-Cog-12 score from 0 to 80. The ADAS-Cog-12 allows for differentiation between a subject with mild cognitive impairment (“MCI”) and a subject with Alzheimer's disease, and between different MCI subtypes.

ADAS-Cog-13 includes all ADAS-Cog-11 items as well as a test of delayed word recall and a number cancellation or maze task. ADAS-Cog-13 scores range from 0 to 85. The ADAS-Cog-12 allows for differentiation between a subject with MCI and a subject with Alzheimer's disease, and between different MCI subtypes.

ADAS-Cog-14 includes all ADAS-Cog-11 items as well as delayed word recall, maze, and digit cancellation tasks. ADAS-Cog-13 scores range from 0 to 85.

In the context of the invention, the improvement in motor function may be ascertained by a test known to one of ordinary skill in the art, including, e.g., by MDS Unified Parkinson's Disease Rating Scale (MDS-UPDRS).

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to a subject (e.g., human). Multiple techniques of administering a compound exist in the art including, but not limited to, intravenous, oral, aerosol, parenteral, ophthalmic, pulmonary, and topical administration.

As used herein, the term “co-administering” refers to a compound of Formula (I), (II), (III) and/or (IV) which is administered to a mammal (e.g., a human subject or human patient) together with an appropriate PDE inhibitor (e.g., a PDE 5 inhibitor) so that the two classes of agents provide an overlapping effect. While in certain cases it may be possible to administer the two classes of agents in a single dosage form, it is contemplated that these agents may be separately administered either via the same route of administration or different routes of administration to achieve overlapping effects, taking into account their differing physical/chemical properties (including but not limited to solubility, bioavailability, half-life, metabolism, and clearance/elimination from the body, etc.).

“Pharmaceutically acceptable” refers to a material(s) which are compatible with the activity of the compound useful within the invention, and which are physiologically acceptable to the patient (e.g., human) from a pharmacological/toxicological point of view and to the manufacturing pharmaceutical chemist from a physical/chemical point of view regarding composition, formulation, stability, patient acceptance, and bioavailability.

“Pharmaceutical acceptable carrier” refers to a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium, and zinc salts. Acceptable base addition salts also include organic salts made from basic amines such as, for example, N, N′-dibenzylethylenediamine, chloroprocaine, choline, diethanolamine, ethylenediamine, meglumine (N-methyl-glucamine) and procaine. All of these salts may be prepared by conventional means from the corresponding free base compound by reacting, for example, the appropriate acid or base with the corresponding free base.

An “individual”, “patient”, or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term neurologic disease or disorder is meant to include Alzheimer's disease, dementia (e.g., dementia with Lewy bodies, frontotemporal dementia), tauopathy, Parkinson's disease, an alpha-synucleopathy, Prion's disease, Down Syndrome, Huntington's disease, Amyloid Lateral Sclerosis, multiple sclerosis and other neurodegenerative disorders.

The term “synergy” as used herein means that the amount (i.e., dosage) of each agent alone has no effect and is sub-therapeutic as monotherapy but when administered as a combination is therapeutic.

The term “treat” or “treating” as used herein means reducing the frequency with which symptoms are experienced by a subject or administering the combination of agents or compounds to reduce the frequency and/or severity with which symptoms are experienced. As used herein, “alleviate” is used interchangeably with the term “treat.” Treating a disease, disorder or condition may or may not include complete eradication or elimination of the symptom. Thus, the term “treating” encompasses preventing and slowing a condition described herein.

The term “therapeutic” as used herein means a treatment and/or prophylaxis of a condition or disease state as described herein.

The term “alkyl” as used herein refers to a branched or unbranched saturated hydrocarbon group of 1 to 4, 1 to 8, or 1 to 20 carbon atoms, such as methyl, ethyl, n-propyl, isopropyl, n-butyl, isobutyl, t-butyl, pentyl, hexyl, heptyl, octyl, and the like. Examples of cycloalkyl groups include cyclopentyl and cyclohexyl.

The term “alkenyl” as used herein refers to a hydrocarbon group of 2 to 4, 2 to 8, or 2 to 20 carbon atoms and structural formula containing a carbon-carbon double bond.

The term “alkynyl” as used herein refers to a hydrocarbon group of 2 to 4, 2 to 8, or 2 to 20 carbon atoms and a structural formula containing a carbon-carbon triple bond.

The term “aryl” is defined as any carbon-based aromatic group including, but not limited to, phenyl, benzene, naphthalene, anthracene, phenanthrene, pyrene, and benzo[a]pyrene, etc.

The term “substituted aryl” is defined as an aryl group having at least one group attached to the aryl group that is not hydrogen. Examples of groups that can be attached to the aryl group include, but are not limited to, alkyl, alkynyl, alkenyl, aryl, heterocyclic, halide, nitro, amino, ester, ketone, aldehyde, hydroxy, carboxylic acid, alkoxy, cyano, alkoxy, thioalkyl, haloalkyl, hydroxyalkyl, alkylamino, diakylamino, or acyl. In various embodiments, a substituent is bound to carbon 2, 3, 4, 5, or 6 of one of these moieties. Examples of alkoxy substituents include, but are not limited to, methoxy, ethoxy, and isopropoxy groups. Examples of acyl substituents include acetyl and benzoyl groups.

The term “aralkyl” is defined as an aryl group having an alkyl, alkynyl, or alkenyl group attached to the aryl group. An example of an aralkyl group is a benzyl group.

The term “heteroaryl” is defined as an aryl group that has at least one heteroatom such as nitrogen, sulfur, or oxygen incorporated within the ring of the aryl group.

The term “heteroalkyl” is defined as an alkyl group that has at least one heteroatom, such as nitrogen, sulfur, oxygen, or phosphate, incorporated within the alkyl group or attached to the alkyl group.

As used herein, “7a” means “4-[(3-Chloro-4-methoxybenzyl)amino]-8-cyclopropyl-3-(hydroxymethyl) quinoline-6-carbonitrile”.

### BRIEF DESCRIPTION OF THE DRAWINGS

FIG. 1 is a Western blot and a graph showing that buntanetap lowers APP in vitro in a dose-dependent manner in SH—SY-5Y human neuroblastoma cells.

FIG. 2A is a Table, collection of graphs and a Western blot showing that buntanetap treatment of APP/PS1 transgenic AD mice reduced APP and its fragments in hippocampus.

FIG. 2B shows dose-dependent improvement in cognition in the population with confirmed early AD.

FIG. 2C shows that Buntanetap's efficacy is strongly correlated with MMSE status.

FIG. 3 is a table showing the reduction of APP/Abeta, tau/phospho-tau and alpha-synuclein in the spinal fluid of mildly cognitive impaired patients.

FIGS. 4A and 4B are graphs of the results of the electrophysiological study of Example 4.

FIG. 4A shows that subthreshold doses of buntanetap (1 μM) plus 7a (1 nM) rescued the LTP defect of mouse hippocampal slices perfused with 200 nM oligomeric Aβ for 20 min prior to a theta-burst stimulation; whereas she same concentrations of buntanetap (1 μM) and 7a (1 nM) alone did not ameliorate the Aβ-induced LTP defect. FIG. 4B shows fEPSP of the last 5 minutes of the LTP curves shown in FIG. 4A.

### DETAILED DESCRIPTION OF THE INVENTION

The invention is directed in part to a method to inhibit, prevent or treat a neurodegenerative disease such as Alzheimer's disease via the administration of buntanetap, compounds that are similar to buntanetap as described herein (e.g., a drug encompassing Formula (I), Formula (II), Formula (III) or Formula (IV)), pharmaceutically acceptable salts and complexes thereof, together with a PDE inhibitor (e.g. a PDE 5 inhibitor) in the same or different pharmaceutical composition(s). The pharmaceutical composition may include one or more pharmaceutically acceptable excipient(s) in addition to buntanetap and/or the PDE inhibitor.

Buntanetap is a selective inhibitor of amyloid precursor protein (APP) production and has potential utility as a disease modifying treatment for AD (Cullen 2006; Utsuki 2006; Lahiri 2007). Buntanetap was discovered at the National Institute on Aging and was selected from a series of structurally related compounds designed for APP specificity with no or minimal acetylcholinesterase inhibitory activity. Buntanetap was shown to reduce APP and consequently beta-amyloid (Aβ) production in relevant preclinical in vitro and in vivo studies. Maccecchini, et al., “Buntanetap as a Candidate Drug to Lower CSF Amyloid Precursor Protein, Amyloid-β Peptide and τ Levels: Target Engagement, Tolerability and Pharmacokinetics in Humans”, J. Neurosurg. Psychiatry 2012; 83:894-902, hereby incorporated by reference, reported the results of a study of buntanetap single and multiple ascending dose phase 1 randomized, double blind, placebo-controlled safety, tolerance, pharmacokinetic studies were undertaken in 120 healthy human volunteers to define a dose that was then used in a small non-randomized study of five MCI subjects. Buntanetap doses up to 4×60 mg daily×10 days were well tolerated. In plasma buntanetap, at all doses, was absorbed rapidly (Tmax=1.2 to 1.7 h) and cleared from the circulation biphasically (terminal half-life of 4.3-4.7 h). Buntanetap proved well tolerated and significantly lowered CSF levels of sAPPα, sAPPβ, t-tau, p-tau, and specific inflammatory markers, and demonstrated a trend to lower CSF Aβ₄₂. Buntanetap's activity is also described in Applicant's U.S. Pat. No. 10,383,851, hereby incorporated by reference. Phase II data for Buntanetap has been published, C. Fang et al, Buntanetap, a Novel Translational Inhibitor of Multiple Neurotoxic Proteins, Proves to Be Safe and Promising in Both Alzheimer's and Parkinson's Patients, J Prev Alzheimers Dis (2022). https://doi.org/10.14283/jpad.2022.84, published 10 Oct. 2022, hereby incorporated by reference in its entirety. This publication reported the results of a Phase 2a Clinical Study which was a double-blind, placebo-controlled, multi-center study of 14 early AD patients and 54 early PD patients. AD patients were given either 80 mg buntanetap or placebo QD. PD patients were given 5 mg, 10 mg, 20 mg, 40 mg, 80 mg buntanetap or placebo QD. The primary endpoint was safety and tolerability; secondary endpoint is pharmacokinetics of buntanetap in plasma. The buntanetap was well tolerated at safe at doses up to 80 mg in both AD and PD patients. Cmax and AUC increased with dose without evidence for a plateau up to 80 mg QD. Biomarker data indicated a trend in lowering levels of neurotoxic proteins and inflammatory factors and improving axonal integrity and synaptic function in both AD and PD cohorts. Psychometric tests showed statistically significant improvements in ADAS-Cog11 and WAIS coding in AD patients and MDS-UPDRS and WAIS coding in PD patients.

Buntanetap®, developed by QR Pharma, Inc. (now Annovis Bio, Inc.), is a small molecule that lowers soluble APP protein levels through a post-transcriptional mechanism. Buntanetap is also known as (+)-phenserine. Buntanetap is the stereoisomer of (−)-phenserine (−)-N-phenylcarbamoyl eseroline), which reached clinical assessment for AD as an anticholinesterase inhibitor. Phenserine is an AChE inhibitor which was investigated as being suitable as an agent for therapy for cognitive impairments associated with aging and Alzheimer's disease (U.S. Pat. No. 5,409,948). Due to its high cholinomimetic side effects, phenserine failed in 3 phase 3 clinical studies.

As used herein, the term “buntanetap” refers to (3aR)-1,3a,8-trimethyl-1,2,3,3a,8,8a-hexahydropyrrolo[2,3-b]indol-5-yl phenylcarbamate, with the chemical structure shown in Formula IV below, at a chemical purity of at least 90%, preferably at least 95%, at least 98%, at least 99%, at least 99.5%, at least 99.9% or 100%, having the chemical structural as follows:

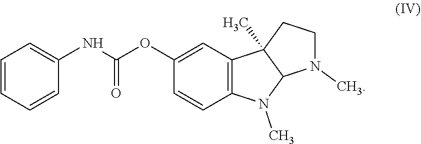 [SMILES: CN1CC[C@]2(C)c3cc(OC(=O)Nc4ccccc4)ccc3N(C)C12]

The term “chemical purity” as applied to (3aR)-1,3a,8-trimethyl-1,2,3,3a,8,8a-hexahydropyrrolo[2,3-b]indol-5-yl phenylcarbamate or a pharmaceutically acceptable salt of buntanetap means the percent by weight of (3aR)-1,3a,8-trimethyl-1,2,3,3a,8,8a-hexahydropyrrolo[2,3-b]indol-5-yl phenylcarbamate or the pharmaceutically acceptable salt of buntanetap in terms of (3aR)-1,3a,8-trimethyl-1,2,3,3a,8,8a-hexahydropyrrolo[2,3-b]indol-5-yl phenylcarbamate or the pharmaceutically acceptable salt of buntanetap and other chemical impurities, e.g., its (−)-enantiomer, that may be present.

The invention also encompasses active metabolites of buntanetap. Active metabolites have previously been identified and include, for example, “N1-nor-Buntanetap” (which refers to (3aR)-3a,8-dimethyl-1,2,3,3a,8,8a-hexahydropyrrolo[2,3-b]indol-5-yl phenylcarbamate) or a salt thereof; “N8-nor-Buntanetap” (which refers to (3aR)-1,3a-dimethyl-1,2,3,3a,8,8a-hexahydropyrrolo[2,3-b]indol-5-yl phenylcarbamate) or a salt thereof; and “N1,N8-nor-Buntanetap” (which refers to (3aR)-3a-methyl-1,2,3,3a,8,8a-hexahydropyrrolo[2,3-b]indol-5-yl phenylcarbamate) or a salt thereof.

In other embodiments, the methods of the present invention are practiced using a phenserine or phenserine-like compound, metabolite, enantiomer, or derivative thereof, known to those skilled in the art, such as those described in U.S. Pat. Nos. 5,171,750; 6,410,747; 6,683,105, 7,153,882; 7,786,162; 7,973,057; 8,258,172; 8,546,430; 8,691,864; and 8,853,253, all of which are incorporated by reference in their entireties.

In a broader sense, the invention is directed in part to the coadministration of a buntanetap-type compound as defined by Formula (I), Formula (II), Formula (III) or Formula (IV) together with a PDE 5 inhibitor drug.

The buntanetap-type compounds include compounds having the Formula I or II as follows:

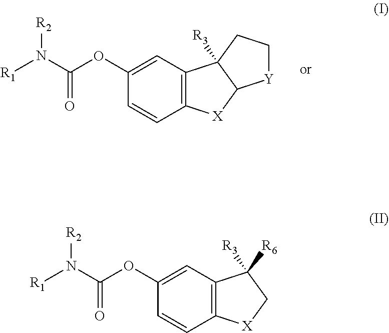

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4, along with an effective amount of a phosphodiesterase inhibitor.

The chiral center of compounds of Formula I and II is the carbon atom that has R₃ bonded to it. As depicted herein, the (+)-enantiomer has R₃ pointing behind the plane of the page. Although only the (+)-isomer is illustrated to save space, in other embodiments the compound having the Formula I or II can be the (+)-isomer, (−)-isomer, and mixtures of both isomers (e.g., racemic mixtures, including 1:1 racemic mixtures) of all of the compounds encompassed by the invention.

In certain embodiments, the compounds having the Formula I or II have an enantiomeric purity for the (+)-enantiomer of from 55 to 100%, desirably from 75 to 100%, more desirably from 85 to 100%, more desirably from 95 to 100%, and even more desirably 100%.

In certain preferred embodiments, wherein the compound having the Formula I or II is the substantially pure (+)-enantiomer.

In one embodiment, when the compound is Formula I, R₃ is methyl and X is NCH₃.

In one embodiment, when the compound is Formula I or II, R₃ is not methyl. In particular embodiments, R₃ is a branched or straight chain alkyl or heteroalkyl group of 2, 3, 4, 5, 6, 7, or 8 carbons or substituted or unsubstituted aryl.

In another embodiment, when the compound is Formula I or II, Y is C(H)R₄ or X is O, S, or C(H)R₄.

In another embodiment, when the compound is Formula I, R₃ is methyl, X is NCH₃, and Y is NCH₃. In one embodiment, when the compound is Formula I, R₃ is methyl, X is NCH₃, Y is NCH₃, and R₁ is C₁-C₈ straight chain alkyl or benzyl and R₂ is hydrogen. In one embodiment, when the compound is Formula I, R₃ is methyl, X is NCH₃, Y is NCH₃, and R₁ is substituted or unsubstituted phenyl and R₂ is hydrogen. In one embodiment, when the compound is Formula I, R₃ is methyl, X is NCH₃, Y is NCH₃, and R₁ and R₂ are, independently, methyl or ethyl.

In another embodiment, when the compound is Formula I, R₃ is methyl, X is NCH₃, and Y is O. In one embodiment, when the compound is Formula I, R₃ is methyl, X is NCH₃, Y is O, R₁ is C₁-C₈ straight chain alkyl or benzyl, and R₂ is hydrogen. In one embodiment, when the compound is Formula I, R₃ is methyl, X is NCH₃, Y is O, and R₁ and R₂ are, independently, methyl or ethyl. In one embodiment, when the compound is Formula I, R₃ is methyl, X is NCH₃, Y is O, and R₁ is substituted or unsubstituted phenyl and R₂ is hydrogen.

In another embodiment, when the compound is Formula I, R₃ is methyl, X is NCH₃, and Y is S. In one embodiment, when the compound is Formula I, R₃ is methyl, X is NCH₃, Y is S, R₁ is C₁-C₈ straight chain alkyl or benzyl, and R₂ is hydrogen. In one embodiment, when the compound is Formula I, R₃ is methyl, X is NCH₃, Y is S, and R₁ and R₂ are, independently, methyl or ethyl. In one embodiment, when the compound is Formula I, R₃ is methyl, X is NCH₃, Y is S, R₁ is substituted or unsubstituted phenyl, and R₂ is hydrogen.

In another embodiment, when the compound is Formula I, R₃ is methyl, X is NCH₃, and Y is NRs. In one embodiment, when the compound is Formula I, R₃ is methyl, X is NCH₃, and Y is NR₅, wherein R₅ is —CH₂CH═CH₂, —CH₂CH₂Ph, benzyl, or hydrogen.

In another embodiment, when the compound has the Formula I, R₃ is methyl, Y is NCH₃, and X is NCH₃, wherein R₄ is benzyl or hydrogen.

In another embodiment, when the compound is Formula I, R₃ is methyl, X is NCH₃, Y is NR₅, wherein each R₄ and R₅ is, independently, hydrogen or benzyl.

In another embodiment, when the compound is Formula I, R₃ is phenyl, X is NCH₃, and Y is NCH₃.

In another embodiment, when the compound is Formula I, R₃ is methyl, and X is NCH₃, and Y is not NH or NHCH₂Ph.

In some embodiments, when the compound is Formula I, R₁ and R₂ are independently, hydrogen, substituted or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ and R₂ are independently, hydrogen or unsubstituted aryl, R₃ is straight chain C₁-C₈ alkyl, X and Y are independently NR₅, wherein R₅ is independently hydrogen or straight chain C₁-C₈.

In some embodiments, when the compound is Formula I, R₁ is hydrogen, R₂ is unsubstituted aryl, R₃ is methyl, X and Y are independently NR₅, wherein R₅ is independently hydrogen or methyl.

In a certain preferred embodiment, when the compound is Formula I, R₁ is hydrogen, R₂ is phenyl, R₃ is methyl, X is NCH₃, and Y is NCH₃.

In other embodiment, when the compound is Formula I, R₁ is hydrogen, R₂ is phenyl, R₃ is methyl, X is NCH₃, and Y is NH.

In a certain preferred embodiment, when the compound is Formula I, R₁ is hydrogen, R₂ is phenyl, R₃ is methyl, X is NH, and Y is NCH₃.

In a certain preferred embodiment, when the compound is Formula I, R₁ is hydrogen, R₂ is phenyl, R₃ is methyl, X is NH, and Y is NH.

In another embodiment, when the compound is Formula II, R₃ is methyl, X is C(H)CH₃, and
R₆ is (CH₂)₂R₇, where R₇ is a substituted or unsubstituted amino group.

In a certain preferred embodiment, when the compound is Formula II, R₃ is methyl, X is NCH₃, and R₆ is (CH₂)₂R₇, where R₇ is a substituted or unsubstituted amino group.

In a certain preferred embodiment, wherein the compound having the Formula II is the substantially pure (+)-enantiomer.

The invention also relates to the use of a compound having the Formula (III) as follows:

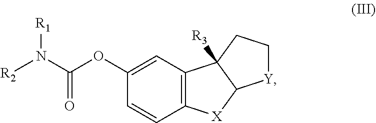

wherein R₁ and R₂ are, independently, hydrogen, branched or straight chain C₁-C₈ alkyl, substituted or unsubstituted aryl, heteroaryl, or aralkyl; R₃ is branched or straight chain C₁-C₄ alkyl or heteroalkyl or C₄-C₈ alkyl or heteroalkyl, or substituted or unsubstituted aryl; X is NR₅, wherein R₅ is C₂₋ₛ alkenyl, C₂₋₈ alkynyl, or aralkyl, and Y is selected from C(H)R₄ or NR₅, wherein R₄ and R₅ are, independently, hydrogen, branched or straight chain C₁-C₈ alkyl or heteroalkyl, alkenyl, or C₂-C₈ alkynyl, aralkyl.

As depicted herein, the (−)-enantiomer has R₃ pointing in front of the plane of the page. Although only the (−)-isomer is illustrated to save space, in other embodiments the compound having the Formula (III) can be the (+)-isomer, (−)-isomer, and mixtures of both isomers (e.g., racemic mixtures, including 1:1 racemic mixtures) of all of the compounds encompassed by the invention.

In a certain preferred embodiment, wherein the compound having the Formula (III) is the substantially pure (−)-enantiomer.

In one embodiment, when the compound is Formula (III), X is NR₅, wherein R₅ is aralkyl.

In one embodiment, when the compound is Formula (III), X and Y are NR₅, wherein R₅ is aralkyl.

In one embodiment, when the compound is Formula (III), wherein X is NR₅, wherein R₅ is aralkyl, and Y is NR₅, wherein R₅ is branched or straight chain C₁₋₈ alkyl or heteroalkyl.

In one embodiment, when the compound is Formula (III), wherein R₁ is branched or straight chain C₁-C₈ alkyl, aralkyl or aryl, R₂ is hydrogen, branched or straight chain C₁-C₈ alkyl, substituted or unsubstituted aryl, or aralkyl; Y is NR₅, wherein R₅ is aralkyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NRs where R₅ is benzyl; and X is NR₅, wherein R₅ is hydrogen, branched or straight chain C₁₋₈ alkyl or heteroalkyl.

In one embodiment, when the compound is Formula (III), wherein R₁ is branched or straight chain C₁-C₈ alkyl, aralkyl or aryl; R₂ is hydrogen, branched or straight chain C₁-C₈ alkyl; Y is NRs where R₅ is benzyl; and X is NR₅, wherein R₅ is hydrogen.

In one embodiment, when the compound is Formula (III), wherein R₁ is para-halophenyl; Y is NCH₃; and X is NR₅, wherein R₅ is alkyl or aralkyl, wherein R₁ is not para-phenyl bromophenyl when R₅ is benzyl.

In one embodiment, when the compound is Formula (III), wherein R₁ is para-isopropyl phenyl; R₂ is hydrogen; R₃ is methyl; Y is NR₅ where R₅ is benzyl; and X is NR₅, wherein R₅ is hydrogen.

Encompassed in the formulations of the invention are the (+)-isomer, (−)-isomer, and mixtures of both isomers (e.g., racemic 1:1 mixtures) of all of the compounds of the invention unless such compounds are specifically excluded.

Variables, such as R₁-R₇, n, X and Y throughout the application are the same variables as previously defined unless stated to the contrary.

The compounds described herein may form salts with acids or bases, and such salts are included in the present invention. In one embodiment, the salts are pharmaceutically acceptable salts. The term “salts” embraces addition salts of free acids or free bases that are compounds of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification, or formulation of compounds of the invention.

Examples of the pharmaceutically acceptable salt of buntanetap include acid addition salts prepared from a suitable acid. The suitable acid can be hydrobromic acid, hydrochloric acid, hydroiodic acid, sulfuric acid, carbonic acid, nitric acid, phosphoric acid, tetrafluoroboronic acid, perchloric acid, 1-hydroxy-2-naphthoic acid, 2,2-dichloroacetic acid, methanesulfonic acid, trifluoromethanesulfonic acid, 2-hydroxyethanesulfonic acid, benzenesulfonic acid, p-toluenesulfonic acid, cyclohexylaminosulfonic acid, 2-oxoglutaric acid, 4-acetamidobenzoic acid, acetic acid, phenylacetic acid, propionic acid, formic acid, succinic acid, glycolic acid, gluconic acid, malic acid, lactic acid, tartaric acid, citric acid, glucuronic acid, ascorbic acid, maleic acid, fumaric acid, pyruvic acid, aspartic acid, glutamic acid, benzoic acid, 4-hydroxybenzoic acid, anthranilic acid, 4-hydroxybenzoic acid, mandelic acid, pamoic acid, pantothenic acid, sulfanilic acid, stearic acid, alginic acid, O-hydroxybutyric acid, salicylic acid, galactaric acid and galacturonic acid. Preferably, the pharmaceutically acceptable salt is buntanetap tartrate, i.e., the acid addition salt of tartaric acid.

Suitable pharmaceutically acceptable base addition salts of compounds of the invention include, for example, metallic salts including alkali metal, alkaline earth metal and transition metal salts such as, for example, calcium, magnesium, potassium, sodium, and zinc salts.

Pharmaceutically acceptable base addition salts also include organic salts made from basic amines such as, for example, N, N′-dibenzylethylene-diamine, chloroprocaine, choline, diethanolamine, ethylenediamine, meglumine (N methylglucamine) and procaine. Examples of pharmaceutically unacceptable base addition salts include lithium salts and cyanate salts. All of these salts may be prepared from the corresponding compound by reacting, for example, the appropriate acid or base.

In certain preferred embodiments, the (buntanetap) compound(s) of the present invention is co-administered with a PDE inhibitor, most preferably a PDE 5 inhibitor drug. Part of the physiological process of vasodilation involves the release of nitric oxide by vascular endothelial cells which then diffuses to nearby vascular smooth muscle cells. There, nitric oxide activates soluble guanylate cyclase which converts guanosine triphosphate (GTP) to cyclic guanosine monophosphate (cGMP), the main effector of the system.

PDE 5 inhibitors are vasodilating drugs that work by blocking the degradative action of cGMP-specific phosphodiesterase type 5 (PDE 5) on cyclic GMP in the smooth muscle cells lining the blood vessels supplying various tissues. PDE 5 inhibitors prolong the action of cGMP by inhibiting its degradation by the enzyme PDE 5, which is found throughout the body. Phosphodiesterase type 5A (PDE 5A) selectively hydrolyzes cyclic GMP. Inhibitors of PDE 5A such as sildenafil are widely used to treat erectile dysfunction, but growing evidence supports important roles for the enzyme in both the vasculature and heart. In disorders such as cardiac failure, PDE 5A upregulation may contribute to a decline in cGMP and protein kinase G signaling, exacerbating dysfunction. PDE 5A plays an important role in the pulmonary vasculature where its inhibition benefits patients with pulmonary hypertension. In the heart, PDE 5A signaling appears compartmentalized, and its inhibition is cardioprotective against ischemia-reperfusion and antracycline toxicity, blunts acute adrenergic contractile stimulation, and can suppress chronic hypertrophy and dysfunction attributable to pressure-overload. In this review, we discuss the molecular biology, pharmacology, and physiology of PDE 5A, mechanisms of vascular and cardiac regulation, and recent evidence supporting the utility of selective PDE 5A inhibition for the treatment of cardiovascular disorders. PDE 5 inhibitors stop a particular enzyme (phosphodiesterase type 5 [PDE 5]), found in blood vessel walls, from working properly. PDE 5 helps control blood flow to the pulmonary arteries. By stopping PDE 5 from working, PDE 5 inhibitors cause the blood vessels to relax. This increases blood flow to the lungs and lowers pulmonary blood pressure. Due to their broad physiological properties, PDE 5 inhibitors are PDE 5 inhibitors are marketed for erectile dysfunction and pulmonary arterial hypertension are undergoing further research in several conditions such as resistant hypertension, myocardial infarction, heart failure, intermittent claudication, Raynaud's phenomenon, chronic kidney disease, and diabetes mellitus. However, to date, the use of PDE 5 inhibitors together with buntanetap or similar compounds has not been suggested.

PDE 5 inhibitors are almost always administered in the form of oral tablets. Therefore, it would be within the abilities of a person skilled in the art to manufacture a combination product containing, e.g., buntanetap or a similar drug as described herein with a PDE 5 inhibitor.

PDE inhibitors were first identified as inhibiting cGMP hydrolysis but not cAMP hydrolysis. In certain embodiments, the PDE 5 inhibitor drug is a nonselective PDE inhibitor which inhibits cGMP-specific PDEs such as PDE 5. An example of a nonselective, weak PDE 5 inhibitor is caffeine. Theophylline and ginseng are also PDE inhibitors. cGMP causes vasodilation in blood vessels by regulating their smooth muscle physiology. It has been shown that the binding of cGMP to the PDE 5 GAF-A domain stimulates enzyme catalytic activity about 10-fold and that blockade of this binding inhibits activity. This suggests that the enzyme is largely inactive in the absence of GAF-A domain ligand binding. Of the 11 families of PDE isozymes (PDE 1-11), what are now identified as PDE 5 and PDE 6 were the first to be found functionally specific for cGMP.

Oral PDE 5 inhibitors commercially available in the U.S. include, e.g., sildenafil (Viagra, Pfizer), vardenafil (Levitra and Staxyn, Bayer/GlaxoSmithKline), tadalafil (Cialis, Eli Lilly), and a more recently approved drug, avanafil (Stendra, Vivus), although mirodenafil, udenafil and lodenafil are available in some countries. Other agents with weak PDE 5 inhibitory properties include zaprinast and icariin. Additional PDE 5 inhibitors not approved, e.g., by the U.S. FDA, have been found as undeclared ingredients or adulterants in a variety of supplements which are sold as “natural” or “herbal” sexual enhancement products. Examples are Acetildenafil, Aildenafil, Homosildenafil, Nitrosoprodenafil, and Sulfoaildenafil. Oral sildenafil lowers pulmonary artery pressures, increases cardiac output, and reduces pulmonary vascular resistance o20% (z30% when combined with inhaled nitric oxide). This occurs with little change in systemic blood pressure, suggesting pulmonary vascular selectivity. In patients with left heart disease and pulmonary hypertension, sildenafil is also now used to assess pulmonary vascular reactivity. Chronic treatment was well tolerated, with the most frequent side effects being headache, dyspepsia, flushing, and diarrhea. More recently, sildenafil has been evaluated to treat PAH from porto-pulmonary, sickle cell, and Gaucher disease.

### Administration and Dosing

In the methods of the invention, buntanetap, its analogs, metabolites, or a pharmaceutically acceptable salt thereof can be administered parenterally or enterally. Examples of the route of administration of buntanetap, or an analog, metabolite, or pharmaceutically acceptable salt or similar compound thereof are intravenous, intraocular, intramuscular, subcutaneous, topical, oral, sublingual, and buccal. Preferably, for purposes of the present invention, buntanetap is administered orally.

In the present invention, buntanetap, or a pharmaceutically acceptable salt of buntanetap, can be administered once, twice, three times, or four times daily. Buntanetap is preferably administered on a once-a-day basis. Depending on the route of administration, buntanetap is administered in different dose ranges.

In certain embodiments (buntanetap) is administered orally in an amount from about 0.01 mg to about 120 mg, preferably on a once-a-day basis. In certain preferred embodiments, buntanetap is administered in an amount from about 0.01 mg to about 10 mg, from about 0.01 mg to about 5 mg, or from about 0.01 mg to about 0.9 mg, preferably on a once-a-day basis.

In certain embodiments, the buntanetap or a pharmaceutically acceptable salt thereof is administered (i) orally in an amount from about 0.1 mg to about 5 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 7 mg/day.

In certain embodiments, the buntanetap or a pharmaceutically acceptable salt thereof is administered (i) orally in an amount from about 0.1 mg to about 5 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 0.7 mg/day.

In certain embodiments, the buntanetap or a pharmaceutically acceptable salt thereof is administered (i) orally in an amount from about 0.01 mg to about 10 mg, from about 0.05 mg to about 5 mg or from about 0.1 mg to about 3 mg on a once-a-day, twice-a-day, three times a day, or four times a day basis; (ii) intravenously in an amount from about 0.01 mg to about 0.3 mg/day; or (ii) intraperitoneally/intramuscularly (IP/IM) in a dose from about 0.03 to about 7 mg/day.

In certain embodiments, the compounds are administered in accordance with the formulations and methods of the invention to a patient who is already presenting with symptoms of a neurological disease. In other embodiments, the compounds are administered in accordance with the formulations and methods of the invention to a patient who is already presenting with pulmonary hypertension. In yet other embodiments, the compounds are administered in accordance with the formulations and methods of the invention to a patient (human subject) who is not presenting with any neurological or hypertensive problems. In certain preferred embodiments, buntanetap is administered orally in a dose from about 0.01 mg to about 5 mg. In other embodiments, the buntanetap dose is administered intravenously in an amount from about 0.01 to about 3 mg/day. In other preferred embodiments, the buntanetap dose is administered intraperitoneally/intramuscularly (IP/IM) in a dose from about 0.03 to about 7 mg/day

In certain embodiments of each of the methods of the present invention as described above, the oral pharmaceutical composition includes from about 0.01 mg to about 10 mg, from about 0.05 mg to about 5 mg, from about 0.1 mg to about 5 mg, from about 0.25 mg to about 5 mg, or from about 0.1 mg to about 5 mg buntanetap or a pharmaceutically acceptable salt thereof, the IP/IM pharmaceutical composition includes from about 0.03 to about 7 mg buntanetap or a pharmaceutically acceptable salt thereof, and the intravenous (IV) pharmaceutical formulation includes from about 0.01 to about 3 mg buntanetap or a pharmaceutically acceptable salt thereof.

In general, the dose of buntanetap preferred to be administered to healthy human patients is a tolerable dose, i.e., a dose that does not cause untoward side effects in a majority of human patient, which dose is also effective for prophylactic treatment of the healthy human(s) with respect to, e.g., neurodegenerative diseases, cancer, cardiovascular homeostasis, diseases or conditions of vital organs, cardiovascular disease, and the like.

In certain preferred embodiments of the methods described herein, peak plasma circulating levels of buntanetap in humans range, e.g., from about 0.1 ng/mL to about 40 ng/mL, in certain embodiments from about 0.2 ng/mL to about 2 ng/mL, and more preferably from about 0.3 ng/mL to about 12 ng/mL. In certain preferred embodiments, the peak plasma circulating level is reached within about 6 hours after administration of buntanetap to humans. In certain embodiments, the peak plasma circulating level is reached within about 3 hours after administration of buntanetap to the humans. In certain embodiments, the plasma circulating level of buntanetap is equal to or greater than about 0.01 ng/mL, 0.05 ng/mL, 0.1 ng/mL, 0.2 ng/mL, 3 ng/mL, 4 ng/mL, 5 ng/mL, 6 ng/mL, 7 ng/mL, 8 ng/mL, 9 ng/mL, 10 ng/mL, 11 ng/mL, 12 ng/mL, 13 ng/mL, 14 mg/mL, 15 ng/mL, 16 ng/mL, 17 ng/mL, 18 ng/mL, 19 ng/mL, or 20 ng/mL for at least 9 hours, and preferably for at least 12 hours, after administration of buntanetap to humans. In certain embodiments, the half-life of buntanetap in cerebrospinal fluid after administering is about 12 hours, and the half-life of buntanetap in plasma after administering is about 5 hours.

The therapeutic agent(s) used as the buntanetap-type drug and a phosphodiesterase inhibitor (e.g., PD5 inhibitor) in the formulations and treatments of the present invention are preferably dosed in therapeutically effective amounts known to those skilled in the art. In certain embodiments, the therapeutically effective amount is an amount that yields a maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. In other embodiments, the therapeutic amount of a phosphodiesterase inhibitor (e.g., a PDE 5 inhibitor) drug is subtherapeutic (meaning that the dose of the phosphodiesterase inhibitor is lower than the lowest amount approved (e.g., by a governmental regulatory agency such as the U.S. FDA) for treating hypertension or a lower dose as compared to that drug being administered without the buntanetap-type drug). One skilled in the clinical and pharmacological arts will be able to determine a therapeutically effective amount through routine experimentation, namely by monitoring a subject's response to administration of the agent and adjusting the dosage accordingly. In certain embodiments, the PDE inhibitor is administered to the patent at appropriate time intervals (e.g., concurrently with the buntanetap) and via an appropriate route of administration (e.g., oral, subcutaneous, intravenous, intramuscular). In certain embodiments, the PDE inhibitor is administered together with the buntanetap-type drug in a single formulation where possible.

In certain embodiments, the phosphodiesterase inhibitor is administered in a subtherapeutic dose.

In certain embodiments, the PDE inhibitor is preferably a PDE 5 inhibitor. Preferably the PDE 5 inhibitor is specific for targeting PDE; however, nonspecific PDE 5 inhibitors may be used in accordance with the present invention as well.

The dosing of PDE 5 inhibitors is dependent on the particular PDE 5 inhibitor chosen for use with, e.g., buntanetap. For example, sildenafil's (Viagra®) effects usually last about 4 hours but can last as long as 12 hours. In some of the embodiments, sildenafil is administered in doses between 2.5 to 100 milligrams (mg) per day. Tadalafil (Cialis®) has the longest action of any available PDE 5 inhibitor at 24 to 36 hours. In some of the embodiments, tadalafil is administered daily at a dose from about 0.25 to 20 mg. Vardenafil (Levitra®) has the shortest onset of action of any currently marketed PDE 5 inhibitor. It usually lasts for about 5-7 hours but may last for up to about 12 hours. In certain embodiments, vardenafil is dosed at 0.5 to 20 mg per day. Avanafil (Stendra®) has a longer duration of activity than vardenafil and sildenafil but shorter than tadalafil. It has a half-life of about 5 hours and, in certain embodiments, is administered in a dose of from 5 to 200 mg per day. All of these doses are oral doses. All of these doses are oral doses.

The oral dose of PDE 5 inhibitors recommended for the treatment of pulmonary arterial hypertension (PAH) is sildenafil 20 mg three times a day or tadalafil 40 mg once a day. In some of the embodiments of the present invention, sildenafil and tadalafil are administered at daily doses that do not exceed 5 mg/day.

In addition to the above, it is known to those skilled in the art that PDE 5 inhibitors are also found in dietary supplements. Examples of such agents include Kaempferia parviflora (black ginger), Tribulus terrestris, Malculra pomifera, and combinations of any of the foregoing.

As previously mentioned, the dose of PDE inhibitor (e.g., PDE 5 inhibitor) may be as set forth above or at a lower dose that would be considered subtherapeutic if being administered for erectile dysfunction.

### Pharmaceutical Compositions and Therapies

Administration of compounds useful within the invention may be achieved in a number of different ways, using methods known in the art. The therapeutic and prophylactic methods of the invention thus encompass the use of pharmaceutical compositions comprising the compounds useful within the invention to practice the methods of the invention. The pharmaceutical compositions of the invention may comprise compounds (1) and (2) formulated and administered in a pharmaceutical formulation together with one or more pharmaceutically acceptable excipients.

Alternatively, the pharmaceutical compositions may be comprised of one of compounds (1) or (2) together with one or more pharmaceutically acceptable excipient. In such embodiments, compound (1) and (2) are administered separately but such that they provide overlapping therapeutic effects.

The relative amounts of the active ingredients, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Typically, buntanetap dosages which may be administered in a method of invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 μg to about 10 mg per kilogram of body weight of the animal. Preferably, the dosage will vary from about 3 μg to about 30 mg per kilogram of body weight of the animal.

Pharmaceutical compositions that are useful in the methods of invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses of, e.g., buntanetap and the phosphodiesterase inhibitor medication. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The composition of the invention may consist of the active ingredient alone, in a form suitable for administration to a (human) subject or patient, or the composition may comprise at least one active ingredient and one or more pharmaceutically acceptable excipients.

In one embodiment, the compositions of the invention are formulated using one or more pharmaceutically acceptable carriers. Pharmaceutically acceptable carriers that are useful, include, but are not limited to, glycerol, water, saline, ethanol, and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The carrier may be a solvent or dispersion medium containing, for example, water, ethanol, polyol (for example, glycerol, propylene glycol, and liquid polyethylene glycol, and the like), suitable mixtures thereof, and vegetable oils.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an antioxidant and a chelating agent that inhibits the degradation of the compound. Preferred antioxidants for some compounds are BHT, BHA, alpha-tocopherol, and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing, or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredients in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Controlled- or sustained-release formulations of a composition of the invention may be made using conventional technology, in addition to the disclosure set forth elsewhere herein. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans-, and perivaginally), (intra)nasal, and (trans)rectal), intravesical, intrapulmonary, intraduodenal, intragastric, intrathecal, subcutaneous, intramuscular, intradermal, intra-arterial, intravenous, intrabronchial, inhalation, and topical administration.

For oral administration, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more inert, non-toxic pharmaceutically excipients. Such excipients include, for example an inert diluent such as lactose; granulating and disintegrating agents such as cornstarch; binding agents such as starch; and lubricating agents such as magnesium stearate. The oral compositions of the invention in the form of tablets or capsules prepared by conventional means with pharmaceutically acceptable excipients such as binding agents; fillers; lubricants; disintegrates; or wetting agents.

Tablets may be non-coated, or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. For oral administration, if desired, the tablets may be coated using suitable methods and coating materials such as OPADRY® film coating systems available from Colorcon, West Point, Pa. (e.g., OPADRY® OY Type, OYC Type, Organic Enteric OY—P Type, Aqueous Enteric OY-A Type, OY-PM Type and OPADRY® White, 32K18400).

Hard capsules comprising the active ingredients may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid preparation for oral administration may be in the form of solutions, syrups, or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters, or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications.

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patent Applications Nos. 20030147952, 20030104062, 20030104053, 20030044466, 20030039688, and 20020051820.

Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### DETAILED DESCRIPTION OF PREFERRED EMBODIMENTS

The following examples further illustrate aspects of the present invention. They are provided for the purpose of illustration only, and the invention is not limited to these examples, but rather encompasses all variations that are evident as a result of the teachings provided herein.

### Example 1

Three Phase I clinical studies have established buntanetap's safety. The pharmacokinetic analyses have demonstrated that the small lipophilic molecule readily enters the brain where its concentration is about 8 times higher than in plasma. Importantly, buntanetap normalized levels of APP, Tau, and αSYN in the cerebrospinal fluid (CSF) of MCI subjects at a dose of 4×60 mg/day. Buntanetap had a >12 h half-life in CSF of MCI subjects, and its effect in lowering these neurotoxic proteins and inflammation extended throughout the 12 h sampling period after the last dose (Maccecchini, et al., “Buntanetap (Posiphen) as a Candidate Drug to Lower CSFAmyloid Precursor Protein, Amyloid-β Peptide and τ Levels: Target Engagement, Tolerability and Pharmacokinetics in Humans”, J. Neurosurg. Psychiatry 2012; 83:894-902). Therefore, we conclude that a much lower single daily buntanetap dose would be effective in the proposed study. In fact, much lower doses were studied in a double Alzheimer/Parkinson phase 2 study that was conducted and completed in 2021(effective IND #72,654). The double phase 2 clinical trial recruited 14 AD (Alzheimer's disease) and 54 patients and treated them over 25 with a once daily dose of buntanetap. The 14 AD patients receive either 80 mg QD or placebo, whereas the 54 PD (Parkinson's disease) patients received 5, 10 20, 40 80 mg QD or placebo. In a nutshell the data shows that in AD and in PD patients (a) buntanetap crossed the blood brain barrier, (b) reduced neurotoxic protein biomarkers, (c) reduced inflammatory markers, (d) improved axonal and synaptic function, and most importantly improved the affected function in both patient populations. In AD patients, buntanetap improved cognition as measured by ADAS-Cog11 and WAIS coding speed (achieving statistical significance versus baseline at 80 mg dose but not placebo). In PD patients it improved at all doses motor function as measured by MDS-UPDRS (Part II, III, IV and total) with the maximum improvement for 10 & 20 mg and improved WAIS speed and accuracy (achieving statistical significance versus placebo in the 5 mg, 20 mg and 80 mg dose arms [p<0.05] of the broader study population [n=54] with the total for all doses also reporting statistically significant improvement [p<0.001]). The data demonstrates the potential benefits of reducing the overexpression of neurotoxic aggregating proteins on inflammation, axonal and synaptic function, and cognitive and functional health. We expect a larger sample population will allow buntanetap to fully demonstrate statistically significant cognitive and functional improvement resulting from the normalization of toxic protein levels in the next planned AD study as has already been demonstrated for PD (where a phase 3 trial started in August 2022).

Buntanetap's effect on neurotoxic proteins. The drug lowers levels of APP in vitro in neuroblastoma cells (Mikillineni et al: Parkinson's Disease; Volume 2012, Article ID 142372, 13 pages. The Anticholinesterase Phenserine and Its Enantiomer Buntanetap as 5′ Untranslated-Region-Directed Translation Blockers of the Parkinson's Alpha Synuclein Expression). It also lowers levels of APP and all its fragments in APP/PS1 transgenic mice (A. F. Teich, et al., Alzheimer's & Dementia: Translational Research & Clinical Interventions 4 (2018) 37-45).

Buntanetap also lowers tau in vitro (Peter Davies Laboratory, Hofstra University, unpublished observation) and in vivo in Human tau mice (Peter Davies Laboratory, Hofstra University, unpublished observation). Buntanetap further lowers αSYN in vitro in neuroblastoma cells and in vivo in transgenic Parkinson's animals in the brain and in the gut (Kuo et. al. Am J Neurodegener Dis 2019; 8(1):1-15 www.AJND.us/ISSN:216 591X/AJND0086080: Translational inhibition of a-synuclein by Buntanetap (posiphen) normalizes distal colon motility in transgenic Parkinsons mice).

Buntanetap's efficacy in treating neurodegeneration in animal models: Restored memory and learning in an APP/PS1 transgenic (tg) mouse model of AD; Restored memory and learning in a Ts65dn mouse model of Down syndrome (DS) [W. Mobley, UCSD, submitted 2020]; Preserved the retina in acute glaucoma [J Sundstrom; Hershey Medical School]; Restored colonic motility in a human SNCAA53T tg mouse model of PD (Kuo et. al., Am J Neurodegener Dis 2019; 8(1):1-15 www.AJND.us/ISSN:216 591X/AJND0086080: Translational inhibition of a-synuclein bhy Buntanetap normalizes distal colon motility in transgenic Parkinson mice); Preserved memory and learning in traumatic brain injury rats (M-F Chesselet, submitted 2020).

Buntanetap's reversal of the neurotoxic cascade: buntanetap's mechanism of action is related to APP, Tau, and αSYN expression being regulated by IRP1 and by iron and the way these proteins contribute to neurodegeneration by accumulating as toxic aggregates that impair axonal transport and synaptic transmission, causing inflammation, and, finally, leading to nerve cell death (as described previously). By reducing APP, Tau, and αSYN levels, buntanetap treatment prevented this toxic cascade. In support of this hypothesis, it has been shown that buntanetap: Normalized anterograde and retrograde vesicle transport in fully differentiated Down syndrome nerve cells [W. Mobley; USCD]; Normalized impaired synaptic transmission in rat striatum after traumatic brain injury (TBI) [M-F Chesselet; UCLA] and hippocampus of APP/PS1 tg mice; lowered inflammation in human CSF of MCI subjects and in the rat brain after TBI; protected nerve cells in rat substantia Ingra after TBI and in a rat acute glaucoma model (J Sundstrom; Hershey Medical School).

The AD field has been dominated by approaches to prevent APP processing or remove Aβ in one of its many forms. These are downstream targets; buntanetap prevents the translational synthesis of the two main proteins involved in AD-APP and tau- and hence should remove all the downstream consequences produced by these proteins. Similarly, the PD field mostly focuses on inhibiting accumulation of αSYN aggregates and the effect of other proteins in this pathway, including LRRK or Parkin. Again, buntanetap prevents the synthesis of αSYN and thus it should stop the pathological cascade at the first step. Our data indicate that by normalizing the levels APP/Aβ, Tau/phospho-Tau, and αSYN, buntanetap normalizes axonal transport, lowers inflammation, and protects nerve cells from dying. (Mobley 2020, submitted for publication; Chesselet 2020, submitted for publication).

### Example 2A

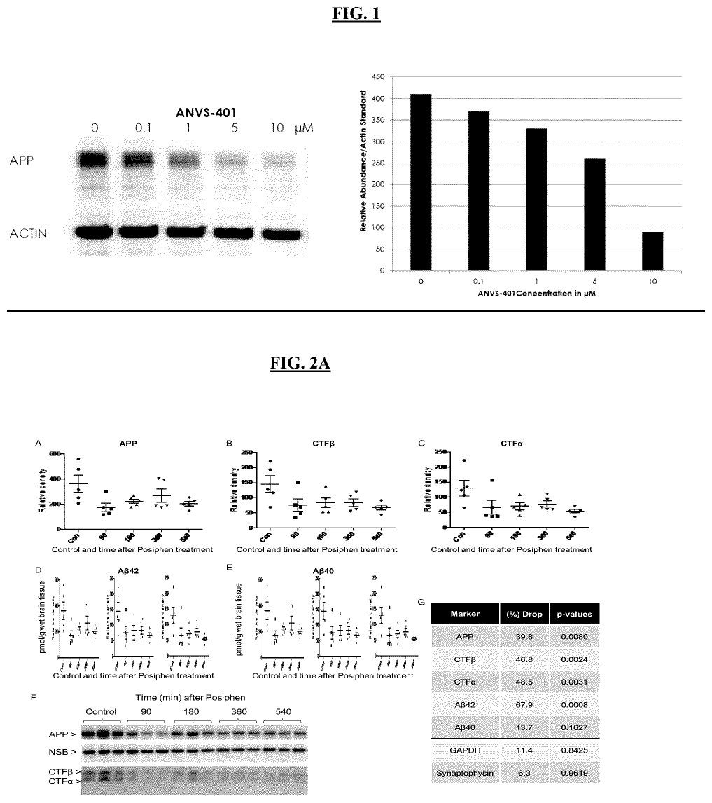

FIG. 1, APP in vitro. shows that buntanetap lowers APP in vitro in a dose-dependent manner in SH—SY-5Y human neuroblastoma cells. On the left is a Western blot showing buntanetap inhibition of APP in relation to Actin standard at concentrations of 0, 0.1, 1, 5 and 10 μM and a graph showing the same data plotted for statistical analysis purposes.

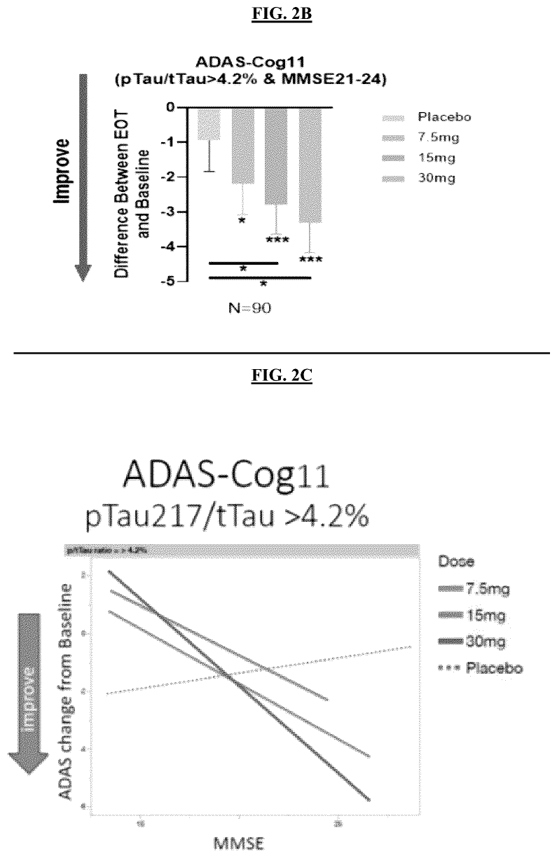

FIG. 2A, APP in vivo. This study was conducted to demonstrate the effect of buntanetap in inhibiting the translation of APP and its fragments in an AD model in vivo. The Table in FIG. 2 shows that buntanetap treatment of APP/PS1 transgenic AD mice reduced APP and its fragments in hippocampus. GAPDH and Synaptophysin were loading controls. FIG. 2 also includes a collection of graphs showing relative density of APP plotted against control and time after buntanetap treatment; relative density of CTFβ plotted against control and time after buntanetap treatment; relative density of CTFα plotted against control and time after buntanetap treatment; Aβ42 levels in brain tissue plotted against control and time after buntanetap treatment; and Aβ40 levels in brain tissue plotted against control and time after buntanetap treatment. Finally, FIG. 2 also includes a Western blot showing levels of APP, NSB, CTFβ, and CTFα after buntanetap treatment over time (minutes). In APP/PS1 mice expressing human mutations associated with familial AD, the data show that buntanetap treatment reduced APP and all related peptides in hippocampus for at least 9 hours after the last dose.

### Example 2B

A randomized, double-blind, placebo-controlled Phase II/III study trial investigating the efficacy, safety, and tolerability of buntanetap in patients with mild to moderate AD was conducted. This was a dose-ranging study where patients received either one of three doses of buntanetap (7.5 mg, 15 mg, or 30 mg) or placebo on top of their standard of care for 12 weeks. In this study, over 700 patients were screened, a total of 353 patients were enrolled, and 325 patients completed the study across 54 sites in the US. The study included mild to moderate AD patients whose Mini Mental State Examination (MMSE) scores at baseline ranged from 14 to 24. (www.clinicaltrials.gov (NCT05686044)).

Beyond safety, the trial assessed the changes in two co-primary endpoints: Alzheimer's Disease Assessment Scale-Cognitive Subscale 11 (ADAS-Cog 11) and Alzheimer's Disease Cooperative Study Clinician's Global Impression of Change (ADCS-CGIC), which assess cognition and activities of daily living. The study monitored for safety and collected plasma to measure several biomarkers to assess the disease state, potential disease progression, and treatment effects.

A significantly higher improvement in ADAS-Cog 11 scores in each treatment dose relative to placebo for patients with mild AD was observed. The analysis focused on biomarker-positive early AD patients (MMSE 21-24, pTau217/tTau≥4.2%) found that ADAS-Cog 11 was highly statistically significant at all 3 dose levels and in the combined dose levels compared to placebo as well as to baseline (FIG. 2B). The treatment response in the current study was not related to a patient's age or sex.

At the end of 3 months of treatment, placebo group demonstrated slight improvement (LSM(SE), 0.26 (0.91)), but not significantly different from baseline. However, all three buntanetap treatment groups showed statistically significant improvement from their corresponding baseline (7.5 mg improved 2.19 (0.87), p=0.013; 15 mg improved 2.79 (0.81), p=0.001; 30 mg improved 3.32 (0.82), P<0.001). Both 15 mg and 30 mg treatment groups also had a statistically significant improvement relative to placebo group (p=0.042 and 0.015 respectively). EOT-End of Treatment * P<0.05; ** P<0.01; ***P<0.001.

When the baseline MMSE scores for patients positive for AD according to their pTau217/tTau >4.2% ratio were subdivided, a dose-dependent relationship to MMSE at baseline was observed. It was concluded that the response to buntanetap treatment is more pronounced in mild AD patients than in those with more advanced AD. The response in the 30 mg dose treatment group R2=0.17 (R² or the coefficient of determination), p<0.001, indicates statistical significance of the MMSE score, which was not evident in the placebo group. FIG. 2C confirms the efficacy of buntanetap as previously shown in FIG. 2B.

It was concluded that was a three-fold difference in the proportion of participants who improved in the 30 mg group relative to placebo (Table 1A).

TABLE 1A
Responders vs Non-Responders

| | Responders | | Non-Responders | | |
|---|---|---|---|---|---|
| | (Number & | | (Number & | | Total |
| Dose | Percentage) | | Percentage) | | Number |
| Placebo | 6 | (27.27%) | 16 | (72.73%) | 22 |
| 7.5 mg Buntanetap | 14 | (73.68%)** | 5 | (26.32%) | 19 |
| 15 mg Buntanetap | 18 | (72.00%)** | 7 | (28.00%) | 25 |
| 30 mg Buntanetap | 21 | (87.50%)*** | 3 | (12.50%) | 24 |

**Contrast with Placebo, p value < 0.01,
***Contrast with Placebo, p value < 0.001

It was concluded that the data presented in FIGS. 2B and 2C demonstrates efficacy of buntanetap in early AD patients.

### Example 3

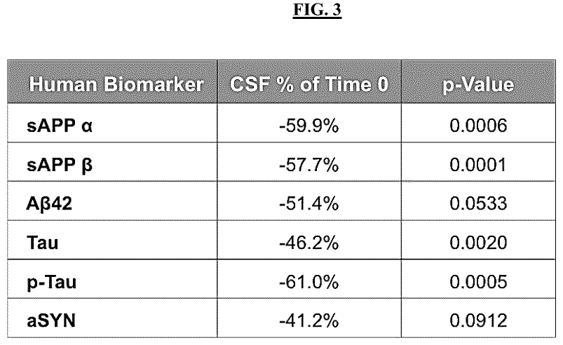

FIG. 3 is a study of subjects with mild cognitive impairment (MCI) for early proof of mechanism (POM) using a well-tolerated dose of buntanetap. Before and after 10 days of buntanetap administration to the MCI subjects, plasma, and cerebrospinal fluid (CSF) samples were obtained for analysis of levels of secreted (s) APPα and APPβ, and Aβ42, Tau (total and phosphorylated), and inflammatory markers. FIG. 3 shows the reduction of APP/Aβ, tau/phosphor-tau and alpha-synuclein in the spinal fluid of mildly cognitive impaired patients. In this study, buntanetap normalized these aggregating proteins in CSF of MCI subjects in accordance with the data seen in animals.

### Example 4

Experiments with Combination of Buntanetap+7a

### Material and Methods

Animals: C57BI6 mice of both sexes were used for these experiments. They were obtained by a breeding colony maintained at the animal facility of Columbia University.

Aβ was prepared as previously described (Stine at al, J Biol Chem. 2003).

### Electrophysiological Analysis

Electrophysiological Analysis was performed on 400 m hippocampal slices, cut with a tissue chopper and maintained in an interface chamber at 29° C. for 90 min prior to recording. Briefly, CA1 fEPSPs were recorded by placing both the stimulating and the recording electrodes in CA1 stratum radiatum. Basal synaptic transmission was assayed, either by plotting the stimulus voltages against slopes of fEPSP, or by plotting the peak amplitude of the fiber volley against the slope of the fEPSP. A 10 min baseline was recorded every min at an intensity that evokes a response, ˜35% of the maximum evoked response in the presence of vehicle. Then slices were either perfused with 200 nM oligomeric Aβ or vehicle with or without compound for 20 min prior to applying a theta burst stimulation (4 pulses at 100 Hz, with the bursts repeated at 5 Hz and each tetanus including 3 ten-burst trains separated by 15 sec). Data was subjected to statistical analyses to determine the effects of combination on inhibiting synaptic dysfunction.

### Statistical Analysis

Slices were coded to blind investigators with respect to treatment. Results were expressed with Standard Error of the Mean (SEM). Level of significance was set for p<0.05. Results were analyzed by ANOVA analysis with post-hoc correction.

It was concluded that buntanetap with the PDE 5 inhibitor 7a protects against amyloid-induced cognitive decline in synaptic plasticity in an ex vivo preparation. Subthreshold doses of buntanetap (1 μM) plus 7a (1 nM) rescued the LTP defect of mouse hippocampal slices perfused with 200 nM oligomeric Aβ for 20 min prior to a theta-burst stimulation; whereas the same concentrations of buntanetap (1 μM) and 7a (1 nM) alone did not ameliorate the Aβ-induced LTP defect (2-way ANOVA: veh vs. Aβ F(1,28)=33.72, P<0.0001; Aβ vs. Aβ+7a 1 nM+buntanetap 1 μm F(1,38)=16.25, P=0.0003; Aβ vs. Aβ+buntanetap 1 μm F(1,31)=2.490, P=0.1247; Aβ vs. Aβ+7a 1 nM F(1,29)=2.415, P=0.1310; Aβ vs. Aβ+buntanetap 10 μM F(1,31)=16.07, P=0.0004; Aβ vs. Aβ+7a 50 nM F(1,37)=12.87, P=0.001; Aβ vs. Aβ+7a 10 nM F_{(1,26)}=5.958, P=0.0218).

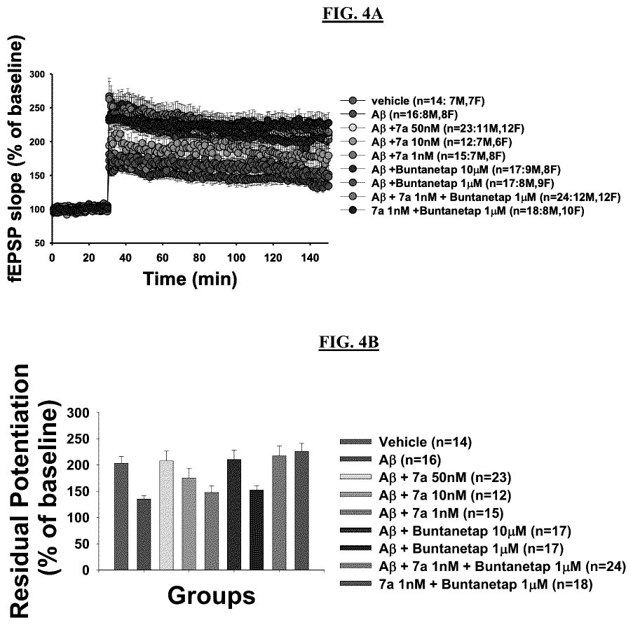

The results are depicted in FIGS. 4A-4B. FIG. 4B is fEPSP of the last 5 minutes of the LTP curves shown in a (1-way ANOVA followed by Bonferroni's comparisons: F_{(8,147)}=4.399 P<0.0001; veh vs. Aβ: P=0.0466; Aβ vs. Aβ+7a 1 nM+buntanetap 1 μm: P=0.001; Aβ vs. Aβ+buntanetap 1 μm: P>0.9999; Aβ vs. Aβ+7a 1 nM P>0.9999; Aβ vs. Aβ+buntanetap 10 μM P=0.0114; Aβ vs. Aβ+7a 50 nM P=0.0082; Aβ vs. Aβ+7a 10 nM P=0.8678). Data means±s.e.m.

The mean values of Residual Potential are provided in Table 2:

TABLE 2

| Treatment Group | Residual Potential |
|---|---|
| veh | 203.04 |
| wt ab | 133.55 |
| ab + 7a 50 nM | 207.77 |
| ab + 7a 10 nM | 174.99 |
| ab + 7a 1 nM | 147.78 |
| ab + buntanetap 1 μM | 152.11 |
| ab + buntanetap 10 μM | 210.49 |
| ab + buntanetap 1 μM + 7a 1 nM | 217.33 |
| buntanetap 1 μM + 7a 1 nM | 225.72 |
| (control) | |

The “veh” represents “normal”, and “wt ab” represents “disease”. The difference between “normal” and “disease” is 69.49 (represents 100% improvement).

The percent improvement in each treatment group, based on the change in the Residual Potential is provided in Table 3:

TABLE 3

| Treatment Group | % Improvement |
|---|---|
| ab + 7a 50 nM | 106.8 |
| ab + 7a 10 nM | 59.63 |
| ab + 7a 1 nM | 14.23 |
| ab + buntanetap 1 μM | 26.7 |
| ab + buntanetap 10 μM | 110.7 |
| ab + buntanetap 1 μM + 7a 1 nM | 120.6 |

It was concluded that the administration of the combination (buntanetap 1 μM+7a 1 nM) provided about 8.5-fold improvement over administration of 7a alone (120.6/14.23=8.48) and about 4.5 from improvement over administration of buntanetap alone (120.6/26.7=4.51).

It was also concluded that the effect provided by the administration of the combination of buntanetap and 7a, as compared to administration of each agent alone, was more than additive (40.93 vs 120.6).

While the invention has been disclosed with reference to specific embodiments, it is apparent that other embodiments and variations of this invention may be devised by others skilled in the art without departing from the true spirit and scope of the invention. All patents and publications cited herein are incorporated by reference in their entirety. The appended claims are intended to be construed to include all such embodiments and equivalent variations.

What is claimed is:

1. A method for treating a neurodegenerative disease comprising co-administration to a human in need thereof:
(1) an amount of a compound selected from the group consisting of Formula (I), Formula (II) and Formula (III): 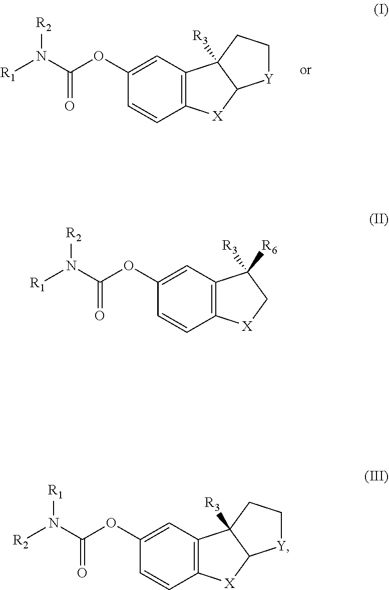
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid, substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof; and
(2) an amount of a phosphodiesterase inhibitor.

2. The method of claim 1, wherein the compound selected from the group consisting of Formula (I), Formula (II) and Formula (III) is buntanetap or a pharmaceutically acceptable salt thereof.

3. The method of claim 1, wherein the phosphodiesterase inhibitor is 4-[(3-Chloro-4-methoxybenzyl)amino]-8-cyclopropyl-3-(hydroxymethyl) quinoline-6-carbonitrile.

4. The method of claim 1, wherein the formulation is administered orally, parenterally, intravenously, subcutaneously, sublingually, via suppository, nasally, topically, transdermally, or via an implant under the skin.

5. The method of claim 4, wherein the human is not demonstrating symptoms of the neurodegenerative disease.

6. The method of claim 4, wherein the human is demonstrating symptoms of the neurodegenerative disease.

7. The method of claim 4, wherein the human is not presenting with erectile dysfunction, pulmonary hypertension and benign prostatic hyperplasia.

8. The method of claim 2, wherein buntanetap or the pharmaceutically acceptable salt thereof is administered (i) orally in an amount from about 1 mg to about 120 mg on a once-a-day basis; (ii) intravenously in an amount from about 0.1 mg to about 25 mg/day; or (ii) intraperitoneally/intramuscularly (IP/IM) in a dose from about 0.3 to about 70 mg/day.

9. The method of claim 2, wherein buntanetap or a pharmaceutically acceptable salt thereof is administered orally in an amount from about 10 mg to about 80 mg on a once-a-day basis.

10. The method of claim 9, wherein the administration provides peak plasma circulating levels of buntanetap from about 1 ng/mL to about 380 ng/mL.

11. The method of claim 1, wherein the compounds are administered in a pharmaceutical formulation together with one or more pharmaceutically acceptable excipients.

12. The method of claim 1, wherein the compounds are administered separately but such that they provide overlapping therapeutic effects.

13. The method of claim 1, wherein the phosphodiesterase inhibitor is a PDE 5 inhibitor.

14. The method of claim 13, wherein the PDE 5 inhibitor is selected from the group consisting of sildenafil, vardenafil, tadalafil, and avanafil, 4-[(3-Chloro-4-methoxybenzyl)amino]-8-cyclopropyl-3-(hydroxymethyl) quinoline-6-carbonitrile, and combinations of any of the foregoing.

15. A pharmaceutical composition, comprising
a compound selected from the group consisting of Formula (I), Formula (II) and Formula (III): 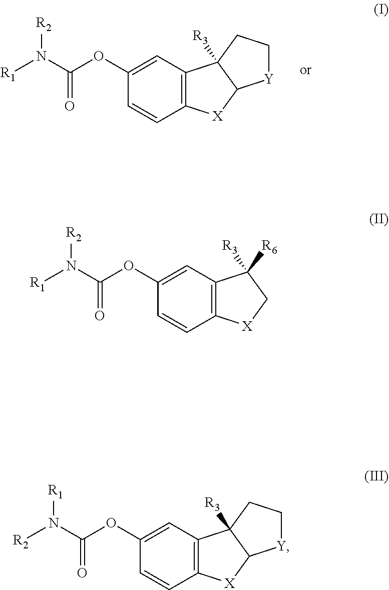
wherein,
in Formula (I) and Formula (II),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X and Y are, independently, O, S, alkyl, hydrocarbon moiety, C(H)R₄, or NR₅, wherein R₄ and R₅ are, independently, hydrogen, oxygen, branched or straight chain C₁-C₈ alkyl, C₂-C₈ alkenyl or C₂-C₈ alkynyl, aralkyl, or substituted or unsubstituted aryl; and
R₆ is hydrogen; C₁-C₈ alkyl, C₁-C₈ alkenyl, C₂-C₈ alkynyl, aralkyl, or substituted or unsubstituted aryl, or (CH₂)ₙR₇, where R₇ is hydroxy, alkoxy, cyano, ester, carboxylic acid,
substituted or unsubstituted amino, and n is from 1 to 4;
wherein,
in Formula (III),
R₁ and R₂ are, independently, hydrogen, branched or straight chain C₁-C₈ alkyl, substituted or unsubstituted aryl, heteroaryl, or aralkyl;
R₃ is branched or straight chain C₁-C₄ alkyl or heteroalkyl or C₄-C₈ alkyl or heteroalkyl, or substituted or unsubstituted aryl;
X is NR₅, wherein R₅ is C₂₋₈ alkenyl, C₂₋₈ alkynyl, or aralkyl;
Y is selected from C(H)R₄ or NR₅, wherein R₄ and R₅ are, independently, hydrogen, branched or straight chain C₁₋₈ alkyl or heteroalkyl, alkenyl, or C₂-C₈ alkynyl, aralkyl and wherein the compound having the Formula (I), Formula (II) or Formula (III) is the substantially pure (−)-enantiomer, the substantially pure (+)-enantiomer, or a racemic mixture of the (−)-enantiomer and (+)-enantiomers or a pharmaceutically acceptable salt thereof;
a phosphodiesterase inhibitor; and
at least one pharmaceutically acceptable excipient.

16. The pharmaceutical composition of claim 15, wherein the compound selected from the group consisting of Formula (I), Formula (II) and Formula (III) is buntanetap or a pharmaceutically acceptable salt thereof.

17. The pharmaceutical composition of claim 16, wherein the phosphodiesterase inhibitor is selected from the group consisting of sildenafil, vardenafil, tadalafil, avanafil, 4-[(3-Chloro-4-methoxybenzyl)amino]-8-cyclopropyl-3-(hydroxymethyl) quinoline-6-carbonitrile, and combinations of any of the foregoing.

18. The pharmaceutical composition of claim 15, which is an oral dosage form.

19. The method of claim 1, wherein the neurodegenerative disease is selected from the group consisting of dementias, tauopathies, chronic traumatic encephalopathy, Parkinson's disease, alpha-synucleopathies, Prion's disease, Down Syndrome, Huntington's disease, Amyloid Lateral Sclerosis, multiple sclerosis, and other dementias and neurodegenerative disorders which present as misfolding, aggregation and accumulation of proteins in the brain.

20. The method of claim 1, wherein the co-administration provides a synergistic therapeutic effect.
